(19) United States
(12) Patent Application Publication
(10) Pub. No.: US 2018/0325820 A1
(43) Pub. Date: Nov. 15, 2018

(54) BIOCOMPATIBLE WATER-SOLUBLE POLYMERS INCLUDING SULFOXIDE FUNCTIONALITY

(71) Applicant: CARNEGIE MELLON UNIVERSITY, Pittsburgh, PA (US)

(72) Inventors: Krzysztof Matyjaszewski, Pittsburgh, PA (US); Sipei Li, Pittsburgh, PA (US)

(21) Appl. No.: 15/980,168

(22) Filed: May 15, 2018

Related U.S. Application Data
(60) Provisional application No. 62/603,001, filed on May 15, 2017.

(51) Int. Cl. A61K 9/14; C08F 293/00; C12N 15/113; A61K 31/713

(52) U.S. Cl. CPC A61K 9/146; C08F 293/005; C12N 2310/14; A61K 31/713; C12N 15/113

(57) ABSTRACT

A biocompatible, water-soluble polymer includes segments formed via an atom transfer radical polymerization and including sulfoxide functionality. The segments including sulfoxide functionality have a dispersity less than or equal to 1.75

### CROSS-REFERENCE TO RELATED APPLICATIONS

This application claims benefit of U.S. Provisional Patent Application Ser. No. 62/603,001, filed May 15, 2017, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Functional water-soluble polymers are desired materials for many bio-medical applications (for example, nucleic acid delivery, such as siRNA delivery, gene delivery, vaccination, tissue engineering, etc.) The utility of such polymers is based in large part on their high in vivo biocompatibility and low cytotoxicity.

Poly(ethylene glycol) (PEG) is among the most widely used polymers in biomedical applications because of its hydrophilicity, water-solubility and chemical stability of the ether groups. Its neutral charge prevents interactions with negatively charged cell membranes, or opsonins, that enhance phagocytosis and polymer clearance. PEGylated species can circulate longer in the bloodstream. PEGylation provides several advantages to the bioconjugates including chemical stability, neutral charge, low cytotoxicity and reasonable water-solubility which generates utility by reducing accessibility for proteolytic enzymes, increasing solubility, reducing immunogenicity and reducing renal clearance issues.

However there are some drawbacks to PEGylated structures including, for example, synthesis of PEG by ionic polymerization and the presence of bi-functional impurities which may result in the formation of heterogeneous products during the conjugation of mono-functional PEG to bio-relevant molecules. Low molecular weight PEG may aggregate at elevated temperatures as a result of its lower critical solution temperature (LCST). In addition, it has been recently reported that the immunogenicity of PEG may result in inefficient drug delivery and severe immune reactions arising from extensive use in cosmetics. These limitations have motivated studies on the preparation and evaluation of other types of water-soluble, biocompatible polymers, with diverse chemistry to replace PEG. Poly(2-oxazoline)s, which are also synthesized by ring-opening polymerization, are potential alternatives to PEG. However, depending on substituents, they may exhibit limited solubility in water.

A number of studies have examined polymers including sulfoxide functionality in forming hydrophilic polymers. For example, 2-(methylsulfinyl)ethyl acrylate (MSEA) has been polymerized via a standard free radical polymerization of the MSEA monomer. The effect of the polymer for cancer treatment in tumor bearing rats was studied. It was noted that the tumor affinity of the polymer was very low, if any.

A different polymer homolog of DMSO, poly(ethylene sulfoxide) has been prepared by selective oxidation of polyethylene sulfide prepared by anionic ring opening polymerization of ethylene sulfide. Others have attempted post-polymerization functionalization formation of a polymeric sulfoxide by modification of incorporated functional groups in, for example, free radical polymerization.

Reversible-Deactivation Radical Polymerization (RDRP; formerly referred to as Controlled Radical Polymerization or CRP) procedures, include, for example, Nitroxide Mediated Polymerization (NMP), Atom Transfer Radical Polymerization (ATRP), and Reversible Addition Fragmentation Transfer (RAFT) and others (including cobalt mediated transfer) that have evolved over the last two decades. RDRP provide access to polymer and copolymers comprising radically polymerizable/copolymerizable monomers with predefined molecular weights, compositions, architectures and narrow/controlled molecular weight distributions. RDRP have emerged as valuable techniques for synthesizing well-defined methacrylate/acrylate-based polymer materials with various architectures including linear polymers, and hybrid materials with inorganic solids or biomolecules.

Neutral oligoethylene glycol acrylate/methacrylate monomers (OEGA/OEGMA) are among the few non-cytotoxic monomers polymerizable by RDRP polymers. However, this type of monomer is difficult be polymerize to high conversion levels as they present high steric hindrance at the growing chin end. It is very desirable to design small neutral water-soluble biocompatible acrylate/methacrylate monomers with low steric-hindrance that could undergo RDRP.

The RDRP procedure, Reversible Addition Fragmentation Chain Transfer (RAFT) Polymerization, has been employed to prepare a series of block copolymer, amphiphilic surfactants with sulfoxide containing hydrophilic monomers. Another RDRP procedure, Atom Transfer Radical Polymerization (ATRP), was indicated to be undesirable because of difficulties in removing catalyst. The RAFT synthesized polymers were subsequently found to lack stability both in aqueous solution and in a dry state.

### SUMMARY

In one aspect, a biocompatible, water-soluble polymer includes one or more segments formed via an atom transfer radical polymerization and including sulfoxide functionality. The segments including sulfoxide functionality have a dispersity less than or equal to 1.75 or less than or equal to 1.3. In a number of embodiments, the segments including sulfoxide functionality may, for example, be formed via an atom transfer radical polymerization of monomer including at least one of 2-(methylsulfinyl)ethyl acrylate or 2-(methylthio)ethyl acrylate. The segments including sulfoxide functionality may, for example, be formed by post-polymerization oxidation of a thio-functional group. One or more of the sulfur atoms in the segments including sulfoxide functionality may be a chiral center.

The segments including sulfoxide functionality may, for example be linear polymer segments or include segments forming branched macromolecules, comb-shaped macromolecules, star macromolecules, nanoparticles, or segments in a gel structure. The polymer may, for example, be a linear polymer, a star shaped polymer, a core/shell macromolecule or a component of a gel.

In a number of embodiments, the segments including sulfoxide functionality are polymerized using an activator regeneration by electron transfer atom transfer radical polymerization process.

In a number of embodiments, the polymer does not exhibit a LCST in aqueous solution below 100° C. Polymers hereof may, for example, be stable when heated to a temperature of 100° C. or even 200° C. in aqueous solution or in a dry state.

The polymer segments including sulfoxide functionality comprises one or more radically copolymerizable monomers other than sulfoxide-containing monomer or thio-containing monomers. The segments may, for example, be block copolymers. The segments may, for example, be random copolymers.

The polymers hereof, may, for example, be attached to a natural biological molecule. The molecule may, for example, include DNA, RNA, a protein or an enzyme.

In another aspect, a method of forming a biocompatible, water-soluble polymer includes forming one or more segments thereof via an atom transfer radical polymerization which include sulfoxide functionality, wherein the segments including sulfoxide functionality has a dispersity less than or equal to 1.75 or less than or equal to 1.3.

The segments including sulfoxide functionality may, for example, be formed via an atom transfer radical polymerization of monomer including at least one of 2-(methylsulfinyl)ethyl acrylate or 2-(methylthio)ethyl acrylate. In a number of embodiments, the segments including sulfoxide functionality are formed by post-polymerization oxidation of a thio-functional group. The segments including sulfoxide functionality may, for example, be formed using an activator regeneration by electron transfer atom transfer radical polymerization process.

In a number of embodiments, the segments including sulfoxide functionality are linear polymer segments or include segments forming branched macromolecules, comb-shaped macromolecules, star macromolecules, nanoparticles, or segments in a gel structure. The polymers and/or segments including sulfoxide functionality may be further characterized as described herein.

In a further aspect, a carrier for delivery a molecule includes a biocompatible, water-soluble polymer including one or more segments formed via an atom transfer radical polymerization and including sulfoxide functionality wherein the segments including sulfoxide functionality have a dispersity less than or equal to 1.75. The polymer may, for example, be a star shaped polymer including the segments, a core/shell macromolecule including the segment or a gel including the segments. In a number of embodiments, the carrier further includes cationic functionality. The segments may, for example, form a hydrophilic shell around a portion including the cationic functionality. The polymer may be further characterized as described herein.

The present systems, methods and compositions, along with the attributes and attendant advantages thereof, will best be appreciated and understood in view of the following detailed description taken in conjunction with the accompanying drawings.

### BRIEF DESCRIPTION OF THE DRAWINGS

FIG. 1. Illustrates an embodiment of a synthetic route to 2-(methylsulfinyl)ethyl acrylate (MSEA) and ¹H NMR spectra of MSEA.

FIG. 2A illustrates kinetics plots of ln([M]₀/[M]) vs time for 1000 ppm Cu^{II} and 100 ppm Cu^{II}(a).

FIG. 2B illustrates plots of Mₙ and M_{w}/Mₙ (dispersity) vs conversion (open circle: Mₙ from GPC at 1000 Cu^{II}, open square: Mₙ from NMR at 1000 Cu^{II}, filled circle: Mₙ from GPC at 100 Cu^{II}, filled square: Mₙ from NMR at 100 Cu^{II}, black dash line: theoretical Mₙ, open triangle: dispersity at 1000 ppm Cu^{II}, filled triangle: dispersity at 100 ppm Cu^{II}).

FIG. 2C illustrates GPC traces at for 100 ppm Cu^{II} and 1000 ppm Cu^{II} ARGET ATRP: condition 1: [MSEA]₀/[EBiB]₀/[CuCl₂]₀:[TPMA]₀:[AA]₀=70/1/0.07/0.15/0.7, 35° C. Condition 2: [MSEA]₀/[EBiB]₀/[CuCl₂]₀:[TPMA]₀:[AA]₀=70/1/0.007/0.015/0.14 (AA added over 5 h), 35° C. (with DMF containing 50 mM LiBr as eluent phase and PMMA as calibration standards)

FIG. 3A illustrates Kelen-Tüdös plots of three monomer pairs.

FIG. 3B illustrates Mayo-Lewis plots of three monomer pairs.

FIG. 4 illustrates activation of H₂O₂ by a chiral confined Bronsted Acid in enantioselective catalytic sulfoxidation.

FIG. 5 illustrates synthesis of an embodiment of star-like polymers hereof.

FIG. 6A illustrates dRI GPC traces of macroinitiator, starMTEAs and starMSEAs.

FIG. 6B illustrates GPC traces of macroinitiator, (polyMTEA)ₙ-polyDVB and (polyMSEA)ₙ-polyDVB.

FIG. 7 illustrates the results of cell toxicity studies of linear polyMSEAs and star-like star MSEAs.

FIG. 8 illustrates grafting of deblock polymer form a structurally tailored and engineered macromolecular or STEM gel.

FIG. 9 illustrates functionalization of a STEM gel hereof.

FIG. 10 illustrates synthesis of an inimer hereof.

FIG. 11 illustrates synthesis of the cationic hyperbranched core with polydimethyl sulfoxide or polyDMSO shell.

FIG. 12 illustrates synthesis of the cationic hyperbranched core with polyOEGMA shell.

FIG. 13 illustrates schematically the relative steric hindrance in core/shell structures with a hyperbranched core and an OEOGMA shell or a MESEMA shell for siRNA complexation.

FIG. 14 illustrates cyclodextrin based star (co)polymers hereof.

FIG. 15 illustrates synthesis of an azide functionalized ATRP initiator.

FIG. 16 illustrates an embodiment of a synthetic route for synthesis of DMSO-Acrylamide.

FIG. 17A illustrates a schematic representation of cationic core-shell nanogels hereof for siRNA delivery and preparation and composition of the nanogels.

FIG. 17B illustrates a size distribution study of the nanogels.

FIG. 17C illustrates a transmission electron microscopy image of one of the nanogels.

FIG. 18 illustrates preparation of STEM gels with distributed —OH functionality.

FIG. 19A illustrates GPC traces of cationic polymer samples (eluent phase: pure DMF at 50° C.

FIG. 19B illustrates GPC traces of cationic hyperbranched core with polyOEGMA shell.

FIG. 19C illustrates GPC traces of ARGET A TRP of MSEMA from cationic core.

FIG. 20A illustrates a photograph of Agarose gel experiments of pure cationic core (SL-7-1-1-12) and polyOEGMA shelled samples (SL-7-1-1-12-1&2).

FIG. 20B illustrates a photograph of Agarose gel experiments of polyDMSO shelled samples (SL-7-1-1-15-7).

FIG. 21 illustrates synthesis of a cationic star polymer here from a cyclodextrin core including grafted quarternized DMAEMA using an ARGET ATRP procedure.

FIG. 22 illustrates the reaction of Thermomyces Lanuginosus (TL) protein in NaPhos buffer with an excess of an amide initiator (2,5-dioxopyrrolidin-1-yl 3-(2-bromo-2-methylpropanamido) propanoate) to modify the protein with a bromide initiator.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “sulfoxide group” includes a plurality of such sulfoxide groups and equivalents thereof known to those skilled in the art, and so forth, and reference to “the sulfoxide group” is a reference to one or more such sulfoxide groups and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

As used herein, the term “polymer” refers to a compound having multiple repeat units (or monomer units) and includes the term “oligomer,” which is a polymer that has only a few repeat units. The term “copolymer” refers to a polymer including two or more dissimilar repeat units or monomer units (including terpolymers—comprising three dissimilar repeat units—etc.).

In a number of embodiments hereof, well-defined, stable, water soluble biocompatible polymers including segments with sulfoxide functional units or sulfoxide functional groups, with controlled architecture are prepared by atom transfer radical polymerization (ATRP). The polymers hereof may, for example, form bio-active conjugates.

Because RDRP processes can provide compositionally homogeneous well-defined polymers, with predicted molecular weight, narrow/designed molecular weight distribution, and high degrees of α- and ω-chain end-functionalization, they have been the subject of much study, as reported in several review articles and ACS symposia. See, for example, Qiu, J.; Charleux, B.; Matyjaszewski, K., Prog. Polym. Sci. 2001, 26, 2083; Davis, K. A.; Matyjaszewski, K. Adv. Polym. Sci. 2002, 159, 1; Matyjaszewski, K., Ed. Controlled Radical Polymerization; ACS: Washington, D.C., 1998; ACS Symposium Series 685. Matyjaszewski, K., Ed.; Controlled/Living Radical Polymerization. Progress in ATRP, NMP, and RAFT; ACS: Washington, D.C., 2000; ACS Symposium Series 768; and Matyjaszewski, K., Davis, T. P., Eds. Handbook of Radical Polymerization; Wiley: Hoboken, 2002, the disclosures of which are incorporated herein by reference. Unfortunately, most current functional water soluble polymers prepared by RDRP-derived procedures, e.g., poly(N,N′-dimethyl ethyl methacrylate)s (PDMAEMAs), poly(N-isopropylacrylamide)s (PNIPAMs), poly(2-hydroxyl ethyl methacrylate)s (PHEMAs), etc., form charged species under physiological conditions and thus become cytotoxic. [J. Controlled Release 2007, 122, 217].

Atom transfer radical polymerization (ATRP) is one of the most powerful and broadly applied procedures [Macromol 1995, 28 (23), 7901; J. Am. Chem. Soc. 1995, 117, 5614; Prog. Polym. Sci. 2001, 26, 337; Chem. Rev. 2001, 101, 2921, the disclosure of which are incorporated herein by reference] among the various RDRP techniques. Normal ATRP, discovered in 1995, generally involved the use of high concentrations of catalysts, therefore efforts were made to reduce the amount of copper. Procedures using lower concentrations of catalyst include ARGET ATRP, [Macromol. 2007, 40, 1789; Angew. Chemie. 2006, 45, 4482] supplemental activator and reducing agent (SARA) ATRP [Macromol. 2013, 46, 8749; Polym. Chem. 2014, 5, 4396; Macromol. 2011, 44, 683] (also termed SET-LRP) [J. Am. Chem. Soc. 2006, 128, 14156, the disclosures of which are incorporated herein by reference] initiators for continuous activator regeneration (ICAR) ATRP, [PNAS 2006, 103 (42), 15309] photoATRP [ACS Macro Letters 2015, 4 (2), 192-196; J. Am. Chem. Soc 2014, 136 (45), 16096] and eATRP. [Science 2011, 332, 81, the disclosure of which is incorporated herein by reference] Noticeably, ATRP has been demonstrated to be efficient in aqueous systems. [J. Am. Chem. Soc 2015, 137, 15430, the disclosure of which is incorporated herein by reference]

The basic ATRP process and a number of improvements to the basic ATRP process have been described in a number of commonly assigned patents and patent applications including, for example, U.S. Pat. Nos. 5,763,546; 5,807,937; 5,789,487; 5,945,491; 6,111,022; 6,121,371; 6,124,411; 6,162,882; 6,624,262; 6,407,187; 6,512,060; 6,538,091; 6,541,580; 6,624,262; 6,627,314; 6,759,491; 6,790,919; 6,887,962; 7,019,082; 7,049,373; 7,064,166, 7,125,938; 7,157,530; 7,332,550; 7,407,995; 7,572,874; 7,678,869; 7,795,355; 7,825,199; 7,893,173; 7,893,174; 8,252,880; 8,273,823; 8,349,410; 8,367,051; 8,404,788; 8,445,610; 8,816,001; 8,865,795; 8,871,831; 8,962,764; 9,243,274; 9,410,020; Ser. Nos. 13/99,3521; 14/239,181; 14/379,418; the disclosures of which are incorporated herein by reference.

ATRP is the most efficient RDRP method for the preparation of pure segmented copolymers, since, generally, unlike RAFT it does not require addition of a standard free radical initiator to continuously form new polymer chains that do not contain the desired α-functional group in a blocking from or a grafting from reaction thereby producing purer segmented or hybrid products. In addition, unlike NMP, ATRP does not require high temperatures to generate the active species by homolytic cleavage of the dormant chain end, which precludes direct formation of bioconjugates, in addition to possessing the capacity to copolymerize a much broader range of radically copolymerizable monomers than NMP.

ATRP allows the synthesis of a, w-homo and hetero-telechelic multi-segmented copolymers with a predetermined degree of polymerization, narrow molecular weight distribution (low M_{w}/Mₙ), incorporating a wide range of functional monomers and displaying controllable macromolecular structures under mild reaction conditions. ATRP generally requires addition or formation of an alkyl halide or (pseudo)halide as an initiator (R—X) or dormant polymer chain end (Pₙ—X), and a partially soluble transition metal complex (Cu, Fe or Ru, for example) capable of undergoing a one electron redox reaction as a catalyst (although metal free ATRP procedures have recently been developed). See, for example, ACS Macro letters 2015, 4, 192-196, the disclosure of which is incorporated herein by reference. The generally accepted mechanism of an ATRP reaction is shown below

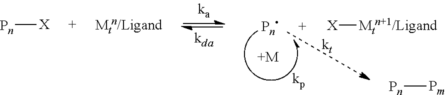

Activator ReGenerated by Electron Transfer (ARGET) ATRP provides an alternative manner of initiating ATRP in which much lower concentrations of catalyst are required. See, for example, Macromolecules, 2012, 45(16): 6371-6379, the disclosure of which is incorporated herein by reference.

The methods of atom transfer radical polymerization (ATRP) may thus yielded rich and diverse polymer architectures. In a number of embodiments, extending chains, side chains or branches of polymers hereof may, for example, be grown/grafted from initiators or from sites of a transfer agent under, for example, aqueous conditions or in the presence of a polar solvent. The extending polymer chains may, for example, be hydrophilic or water soluble. In representative embodiments, the monomers for polymerization/copolymerization may, for example, include sulfur-containing radically polymerizable monomers such as 2-(methylsulfinyl)ethyl acrylate (MSEA) or 2-(methylthio)ethyl acrylate which include or can be modified after polymerization to include one or more sulfoxide groups. Other representative monomers for use herein for, for example, copolymerization with sulfur-containing radically polymerizable monomers include an oligo(ethylene oxide) methacrylate (OEOMA), an ethylene glycol methyl ether acrylate of (OEGA), a di(ethylene glycol) methyl ether methacrylate (MEO₂MA), a 2-(Dimethylamino)ethyl methacrylate, DMAEMA, an acrylamide (AAm), a N,N-dimethylacrylamide (DMA), a N-vinylimidazole (VI), and N-isopropylacrylamide (NIPAM).

Neutral-charged dimethyl sulfoxide (DMSO) is a highly bio-friendly agent. It has been used in a variety of bio-applications, including use as a transdermal enhancer, polymerase chain reaction inhibitor, cryoprotectant, and in veterinary medicines. The high bio-functionality and biocompatibility of DMSO is largely a result of the polar aprotic methyl sulfoxide group. It would be desirable to design functional polymeric materials that possess the inherent advantages of DMSO amplified by the physical and mechanical nature of designed polymeric structures. Such a monomer precursor is 2-(methylsulfinyl)ethyl acrylate or MSEA.

The relative reactivity of MSEA in ARGET ATRP with different monomers (for example, methyl acrylate or MA, nbutyl acrylate of nBA, ethylene glycol methyl ether acrylate of OEGA (OEGA₅₀₀) and methacrylate based monomers) was evaluated by using the Kelen-Tüdös approach. The reactivity ratio of MSEA vs. the three different comonomers by both the Kelen-Tüdös approach and the Fineman-Ross approach provides information on how other functional comonomers may be incorporated into a polyMSEA or PMSEA based polymer with desired distribution of the comonomers. The lower critical solubility temperature (LCST) of the polymers were explored by copolymerization with N-isopropylacrylamide or NIPAM. The extrapolated LCST was greater than 100° C. thereby confirming the suitability of hydrophilic monomers comprising sulfoxide units for bio-applications since they retain their hydrophilic character at all bio-relevant temperatures. The biocompatibility of the synthesized neutral water-soluble linear polyMSEA and starPMSEA polymers both showed low cytotoxicity for human embryonic kidney cells (HEK 293).

Neutral-charged, water-soluble, bio-compatible polymers are of significant importance in bio-medical applications. The synthesis of a polymeric homologue of DMSO is exemplified herein by, but not limited to, (meth)acrylate-based neutral water-soluble monomers containing a sulfoxide groups, exemplified in a number or representative examples by, for example, 2-(methylsulfinyl)ethyl acrylate (MSEA). In a number of studies, well-defined linear polyMSEA was synthesized by activator regeneration by electron transfer atom transfer radical polymerization (ARGET ATRP) with low amounts of copper (100 ppm) and generating polymers with low dispersity (for example, less than 1.75 or less than 1.3). Mono-dispersed star-like polymers incorporating MSEA units were synthesized by ARGET ATRP of 2-(methylthio)ethyl acrylate (MTEA) followed by a chain extension core forming polymerization with a divinyl monomer and subsequent post-oxidation. In other representative examples, well-defined star polymers were prepared in a “grafting from” polymerization from initiator functionalized 3-cyclodextrin. In that regard, star polymers were synthesized by a “core-first” approach using a biodegradable 3-cyclodextrin core and a simple ARGET ATRP polymerization of the desired sequence of comonomers to form well defined stars with either a homopolymeric shell or a segmented polymeric shell using the first formed star as a macroinitiator. Glass transition temperature (Tg) of both linear polymers and star polymers were characterized by DSC.

Unlike prior attempts to prepare polymers comprising sulfoxide groups, the polymer hereof provide well-defined and stable homopolymers and (co)polymers been prepared by an RDRP/ATRP process of a monomer containing a sulfonyl functional group, thereby enabling a simple one step procedure for the synthesis of materials with controlled incorporation of monomers/comonomers and preparation of copolymers with complex architecture designed for targeted bio-applications or for conjugation to biologically active molecules, including, for example, complexation with siRNA. Without limitation to any mechanism, ATRP may provide for synthesis of sulfoxide-containing polymers with increased stability in comparison to other RDRP procedures as a result of increased stability of end groups of the resultant polymers.

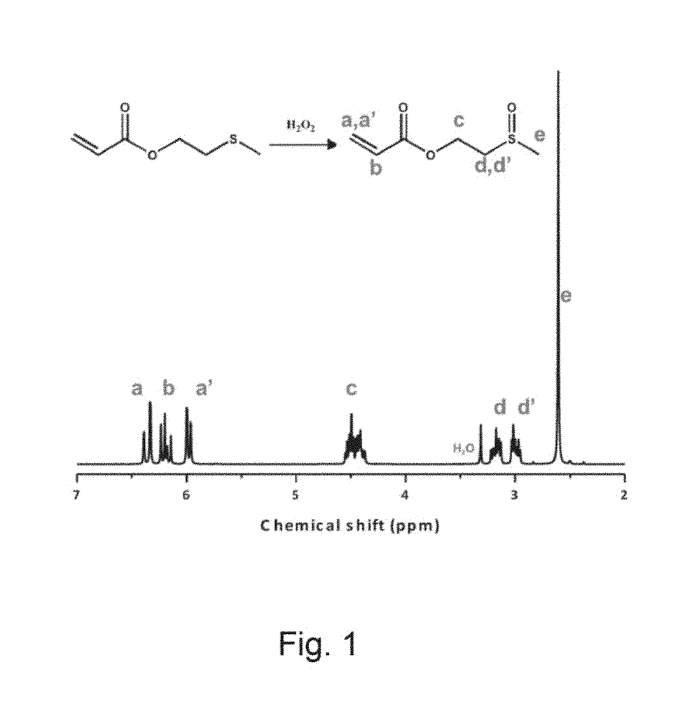

The representative embodiment of synthetic route for MSEA is illustrated in FIG. 1. The first monomer, 2-(methylthio)ethyl acrylate (MTEA), was synthesized by 3-(ethyliminomethyleneamino)-N,N-dimethylpropan-1-amine hydrochloride (EDC) catalyzed esterification. The sulfoxide containing monomer 2-(methylsulfinyl)ethyl acrylate (MSEA) was then synthesized by reacting 1 equivalent of MTEA with 1.1 equivalents of hydrogen peroxide solution. The hydrogen peroxide solution was added to MTEA (liquid state) at a slow feed rate, 50 μL/min, to avoid side-reactions caused by heat release. After the reaction, the raw product was extracted with dichloromethane (DCM) to remove excess hydrogen peroxide.

The ¹H NMR spectrum of MSEA in DMSO-d₆ is also shown in FIG. 1. The fact that the peak integration ratio of a/b/a′/c/d/d′/e=1/1/1/2/1/1/3 indicates the high purity of the synthesized monomer. In a number of representative studies, the monomer was polymerized by ARGET ATRP. The conditions are further described in the experimental section. The employment of ARGET ATRP allows for the use of lower amounts of copper, less than 1000 ppm. In that regard, in an ATRP the polymerization rate depends on the relative ratio of [Cu^{I}-L]/[X—Cu^{II}-L], rather than the absolute amount of copper species (see Eq. 1 below). In a number of studies, ARGET ATRP was performed under two conditions with different amounts of Cu^{II}, either 1000 ppm or 100 ppm. For the 1000 ppm ATRP, all the reducing agent was added at one time at the beginning of the polymerization. For the 100 ppm ARGET ATRP, the reducing agent was added intermittently over 5 h.

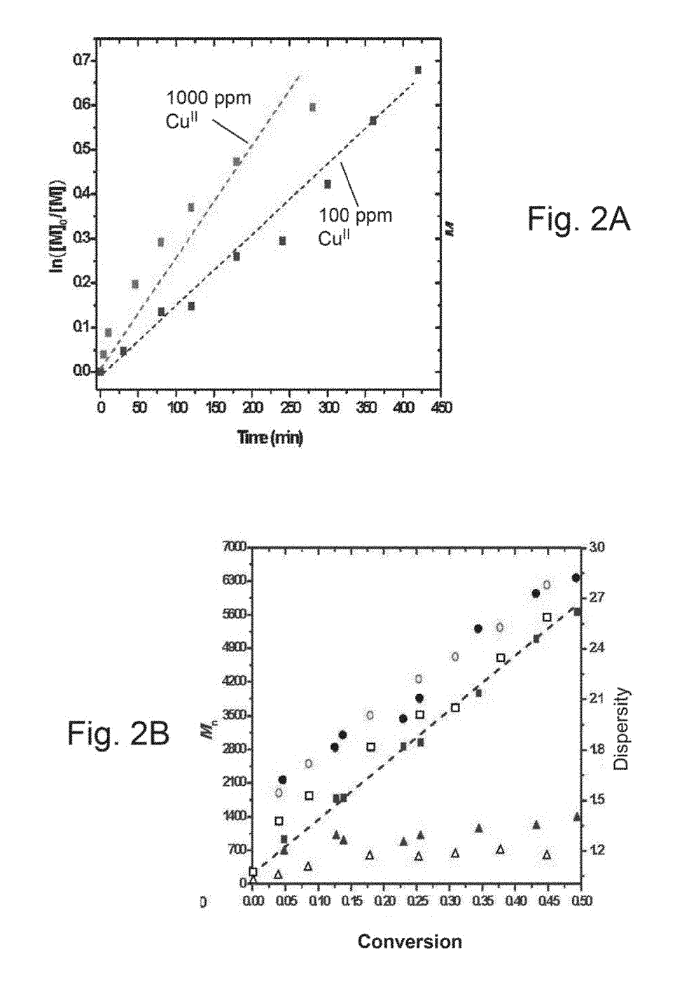

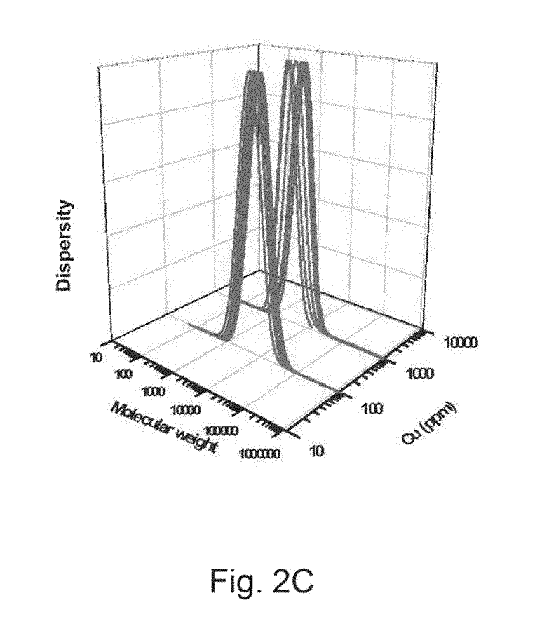

FIG. 2A illustrates the kinetic plots of (ln([M]₀/[M]) vs time). The relatively linear semilogarithmic plot indicates the presence of a steady concentration of active centers during the polymerization. Over time, the MW of polymer formed with 1000 ppm Cu^{II} gradually deviated from the linear trajectory, suggesting that at higher conversion, the retention of chain-end functionality decreased slightly, which may be attributable to catalytic chain termination. In comparison, 100 ppm ATRP with slow feeding of reducing agent showed a better linearity. This phenomenon is agreement with previous results that when a highly active ligand (TPMA in this case) is used, slow feeding of reducing agent tends to minimize termination and allow reaction to reach higher conversion with steadily lower ratio of activator (Cu^{I})/deactivator (Cu^{II}).

FIG. 2B illustrates the change in the number average molecular weight (Mₙ) and dispersity with conversion. The Mₙ from GPC was slightly higher than the theoretical value, which could be a result of a systematic error of the calibration. The Mₙ from NMR was calculated according to Eq. 2 below, where δ_{5.99} represents the integration area of the double bond hydrogen, δ_{4.52} represents the integration area of methylene peaks next to the ester group. δ_{1.16} represents the integration area of methyl groups of EBiB. M_{MSEA} is the molecular weight of monomer MSEA, M_{EBiB} is the molar mass of initiator EBiB. Good agreement between the theoretical value and the M_{n,NMR} indicates good control over the polymerization. FIG. 2B indicates that the dispersity for 1000 ppm Cu^{II} reaction remained below 1.2 during the polymerization. Dispersity for 100 ppm Cu^{II} catalyzed reaction increased slightly as the reaction progressed. The increase in dispersity was a result of the lower actual concentration of deactivator in the polymerization medium, as shown in Eq. 3 below. GPC traces exhibited symmetrical narrow Gaussian distribution for all polymers formed (FIG. 2C), which also suggest good control of the ARGET ATRP.

$$\begin{matrix}{R}_{p} = {k}_{p} [ M ] [ {P}_{n}^{*} ] = {k}_{p } {K}_{ATRP} ( \frac{[ {P}_{n} X ] [ {Cu}^{I} - L ] [ M ]}{⌈ X - {Cu}^{II} - L ⌉} ) & ( 1 ) \\ {M}_{n , NMR} = \frac{3 \times ( \frac{{δ}_{4.52}}{2} {δ}_{5.99} )}{{δ}_{1.16}} \times {M}_{MSEA} + {M}_{EBiB} & ( 2 ) \\ \frac{{M}_{w}}{{M}_{n}} = 1 + \frac{1}{{DP}_{n}} + ( \frac{{k}_{p} [ {P}_{n} X ]}{{k}_{deact} ⌈ X - {Cu}^{II} - L ⌉} ) ( \frac{2}{p} - 1 ) & ( 3 )\end{matrix}$$

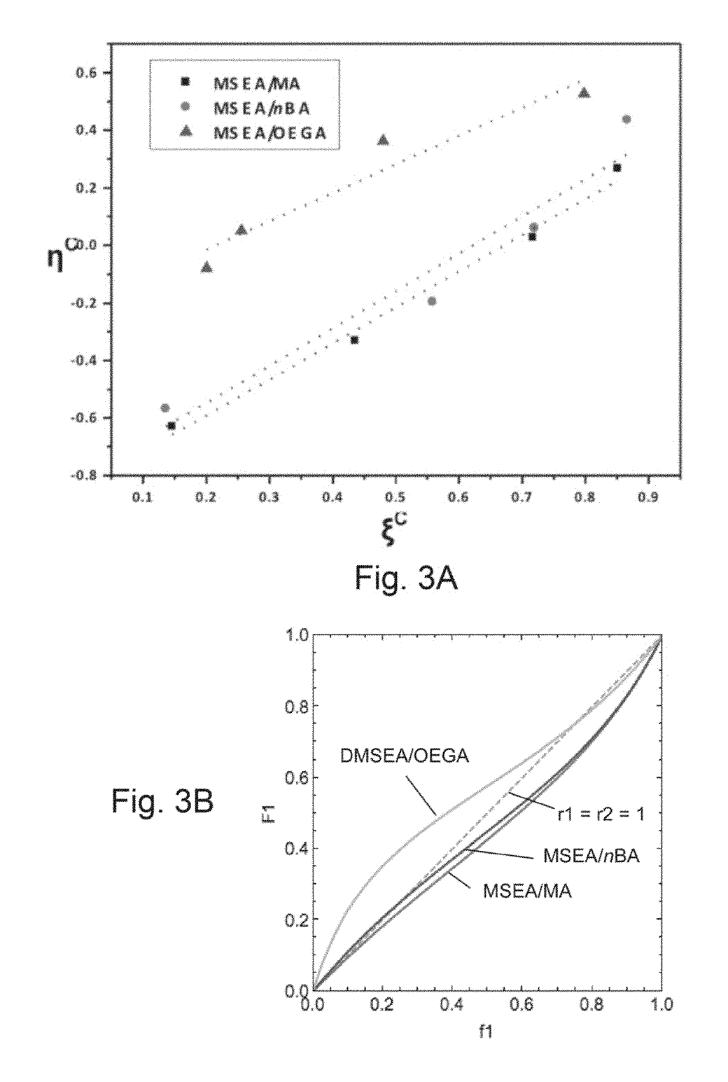

The preparation of functional copolymers is one of the most dynamic areas in polymer industry. The number of commercial copolymers has exceeded that of homopolymers by far. However, the composition of the ultimate copolymer may differ, depending on the reactivity ratios of two monomers. Therefore, the reactivity ratio of MSEA was determined with different comonomers in ARGET ATRP. Three types of monomer pairs were studied by the Kelen-Tüdös approach. They were MSEA/MA, MSEA/nBA and MSEA/OEGA₅₀₀ respectively. The relationship of η^{C} vs. ξ^{C} was plotted according to Eq. 4 below. η^{C} and ξ^{C} were calculated according to Eq. 5 and Eq. 6 below, where m, represent the concentration of polymerized repeating units of monomer i, Mᵢ₀ and Mᵢ represent the initial and final concentrations of monomer i. r₁ and r₂ were calculated according to the slope and intercept of fitted plot in FIG. 3A and Eq. 7. FIG. 3B illustrates the Mayo-Lewis plots, in which f₁ is the initial feed fraction of MSEA, F₁ is the ultimate polymer composition fraction of MSEA. As calculated, for monomer pair MSEA/MA, r₁=0.49, r₂=1.01. For monomer pair MSEA/nBA, r₁=0.49, r₂=0.83. For monomer pair MSEA/OEGA, r₁=0.77, r₂=0.30, in all three pairs, MSEA is monomer 1.

These results indicate, in most cases, the tendency to form copolymers is higher than forming homopolymers and therefore copolymerization with MSEA tends to form random copolymers. Interestingly, for the MSEA/MA pair, the value of r₂ is almost equal to 1, which means when the concentrations of two monomers are the same, polymers with MA radical as chain end have the same possibility to attach either MA or MSEA. The reactivity ratio was also checked by the Fineman-Ross approach, which showed good agreement with the Kelen-Tüdös approach.

$$\begin{matrix}{η}^{C} = ( {r}_{1} + \frac{{r}_{2}}{α} ) {ξ}^{C} - \frac{{r}_{2}}{α} & ( 4 ) \\ {ξ}^{C} = \frac{\frac{{m}_{1}}{{m}_{2}} {( \frac{log ( {M}_{2} / {M}_{20} )}{log ( {M}_{1} / {M}_{10} )} )}^{2}}{α + \frac{{m}_{1}}{{m}_{2}} {( \frac{log ( {M}_{2} / {M}_{20} )}{log ( {M}_{1} / {M}_{10} )} )}^{2}} & ( 5 ) \\ {η}^{C} = \frac{\frac{{m}_{1} - {m}_{2}}{{m}_{2}} ( \frac{log ( {M}_{2} / {M}_{20} )}{log ( {M}_{1} / {M}_{10} )} )}{α + \frac{{m}_{1}}{{m}_{2}} {( \frac{log ( {M}_{2} / {M}_{20} )}{log ( {M}_{1} / {M}_{10} )} )}^{2}} & ( 6 ) \\ α = \sqrt{{( \frac{{M}_{10}^{2}}{{M}_{20}^{2}} \frac{{m}_{2}}{{m}_{1}} )}_{MI N} {( \frac{{M}_{10}^{2}}{{M}_{20}^{2}} \frac{{m}_{2}}{{m}_{1}} )}_{MA X}} & ( 7 )\end{matrix}$$

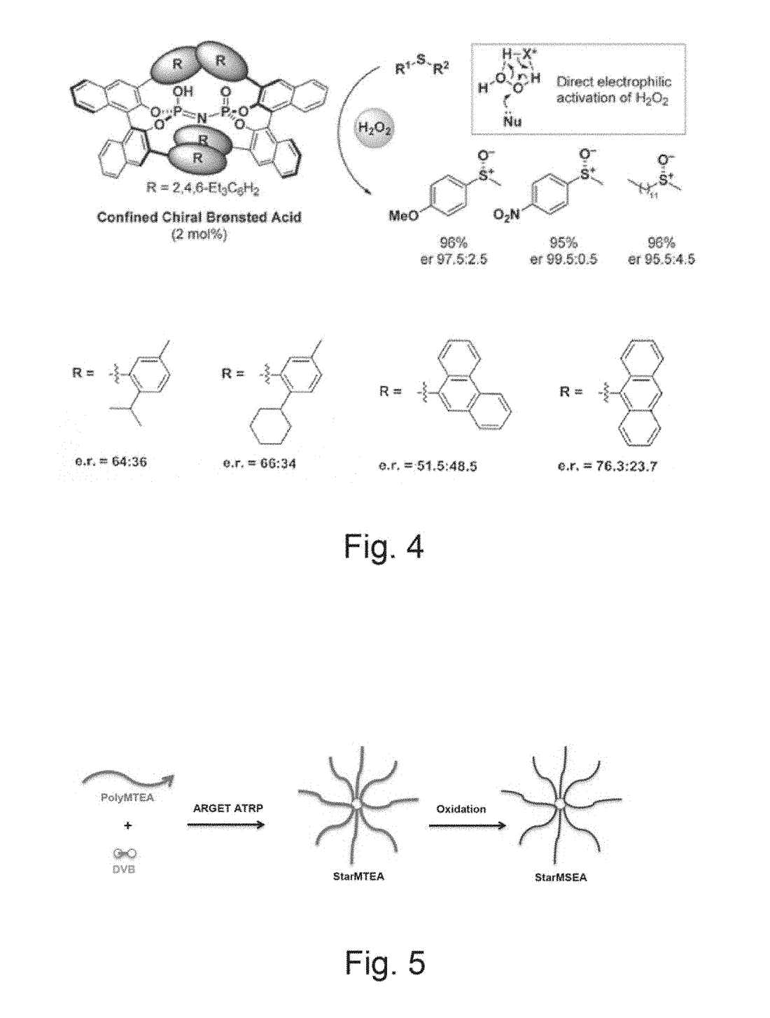

As described above, the monomer for polyDMSO, MSEA, can be synthesized via oxidation of the related thioether with hydrogen peroxide. To make the compound chiral, enantioselective oxidation of sulfur is required. A versatile method using a chiral Bronsted acid to achieve such a transformation is illustrated in FIG. 4 as disclosed in Liao, S. et al, JACS, “Activation of H₂O₂ by Chiral Confined Bronsted Acids: A Highly Enantioselective Catalytic Sulfoxidation,” 2012, 134, 10765-10768, the disclosure of which is incorporated herein by referend. The related thioether precursor was oxidized using the catalyst disclosed in JACS, 2012, 134, 10765-10768. Analysis and separation were performed by chiral HPLC.

Other catalysts were also investigated for acrylate to increase the selectivity. Although suitable for use, the other four catalysts (wherein R for each of the four catalyst is illustrated in FIG. 4) did not give better results. The oxidized sulfoxide was easy to polymerize (for example, under vacuum for a couple of hours). Without limitation to any mechanism, the simplicity of polymerization may arise from the chiral center in the monomer increasing the rate of polymerization. The chiral sulfoxide in solution is much more stable and no polymerization was observed. While replacing catalyst A with catalyst B, in which all ethyl groups on the phenyl ring were replaced by propyl groups, the enatioselectivity was significantly increased. Especially for DMSO-type methacrylate, the enantiomeric ratio (er ratio) was increased to 88:12. Using catalyst C, with pentyl groups, the enatioselectivity was further increased. Oxidation of all three monomers, acrylate, methacrylate, and acrylamide-based monomers, provided good selectivity.

Star-like polymers, can be considered as a type of stable unimolecular micelle, and have potential, for example, as drug-delivery carriers, which can be enhanced for hydrophobic drugs by formation of stars with a more open hydrophobic core. Sulfoxide-containing star polymers (polyMSEA)ₙ-polyDVB were synthesized via a combination of ARGET ATRP and post-oxidation. An embodiment of a synthesis route used in the studies hereof is illustrated in FIG. 5.

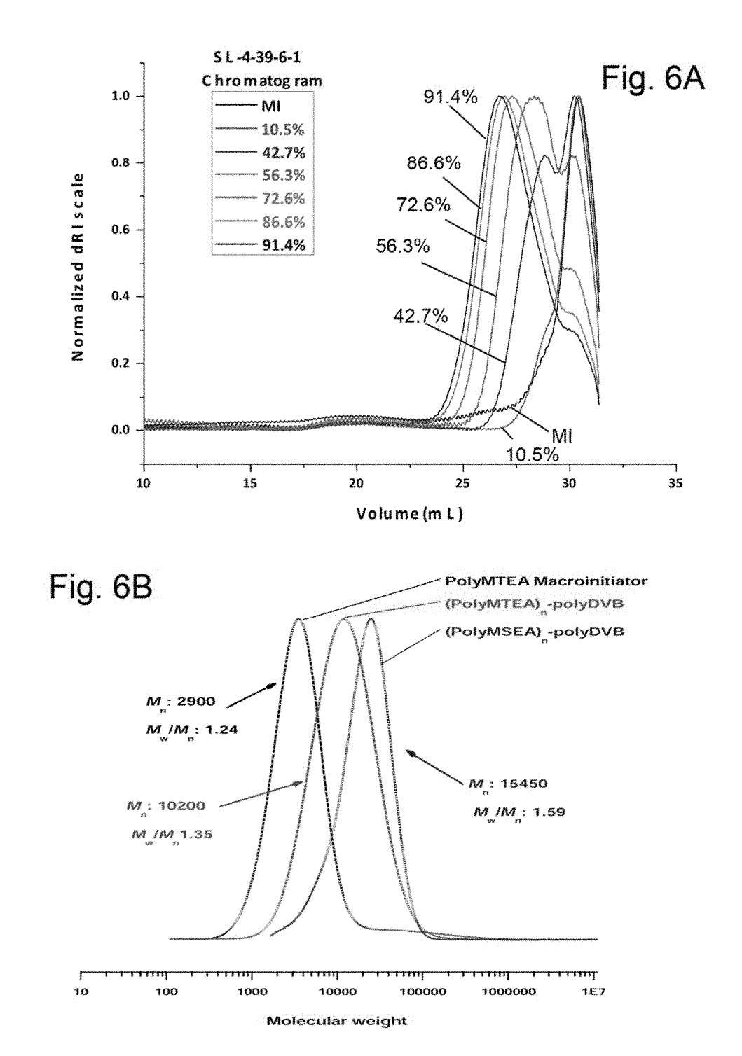

The polyMTEA macroinitiators were synthesized by ARGET ATRP in acetone. The kinetics showed well-controlled polymerizations and the formed polyMTEA polymer with molecular weight of 2,900 and dispersity of 1.24 was used as the macroinitiator for the synthesis of sulfide-containing star-like polymers. The (polyMTEA)ₙ-polyDVB star macromolecules were prepared by ARGET ATRP with Sn(EH)₂ as reducing agent and divinyl benzene as crosslinker. As shown in FIG. 6A, as the reaction proceeded, the macroinitiator peak gradually decreased as the reaction progressed and a new peak at higher molecular weight gradually formed and shifted to higher molecular weight indicating the addition of more arms into the star. The reaction was stopped at 91.4% incorporation of the arm precursors and the star polymer was purified by precipitation and dialysis. The purified (polyMTEA)ₙ-polyDVB showed a monodispersed peak with a molecular weight of 10,200 by dRI detector and 30,500 by light scattering. GPC traces of macroinitiator, (polyMTEA)ₙ-polyDVB and (polyMSEA)ₙ-polyDVB are illustrated in FIG. 6B.

(PolyMSEA)ₙ-polyDVB star polymers were synthesized by post-oxidation of the first formed star with hydrogen peroxide solution and purified by dialysis. After the oxidization to sulfone was complete no further oxidation occurred on continued exposure to hydrogen peroxide indicating the stability of the sulfoxide containing star molecule in the presence of an aqueous oxidation environment. The (polyMSEA)ₙ-polyDVB also showed narrow molecular weight distribution and exhibited a molecular weight of 15,450 by dRI detector and 33,450 by light scattering. The difference between dRI detector and light scattering is attributed to the difference of measurement methods. The dRI detector measures the molecular weight by hydrodynamic volume. However, star-like polymers tend to have smaller hydrodynamic volume than linear polymers with the same molecular weight. Therefore, this difference indirectly confirms the non-linear topology of the star polymer.

In general, the studies hereof demonstrate that well defined star-like polymers of, for example, (polyMSEA)ₙ-polyDVB, with low dispersity may be synthesized by, for example, ARGET ATRP of 2-(methylthio)ethyl acrylate (MTEA) monomer. The formed macroinitiator may be crosslinked by reaction with divinyl benzene (DVB) to form a star macromolecule with subsequent oxidation to form the sulfoxide.

The majority of bio-functional water-soluble polymers typically show a lower critical solubility temperature (LCST), which a result increased hydrophobic interactions at higher temperatures. For example, polyNIPAM has a LCST at ˜32° C. in water, polyDMAEMA has a LCST at ˜50° C. in water, and polyOEGMA has a LCST at ˜90° C. in water. Although the existence of LCST could be utilized to fabricate materials that display special topological properties, this property sometimes limits the scope of their bio-applications because of the decreased solubility of the polymer in water above the LCST. Therefore, the LCST temperature of polyMSEA was studied by dynamic light scattering (DLS). However, in the range of 25˜90° C., direct DLS analysis of pure polyMSEA showed no aggregation. Therefore an extrapolation approach was adopted to study the LCST of polyMSEA. A free-radical copolymerization of NIPAM and MSEA were performed at different molar ratios, see DLS traces for polymers with different compositions. As the content of MSEA increased, the LCST of the copolymer also increased. By extrapolating the LCST to 100% MSEA, the LCST by z-average would be ˜135° C. By extrapolating the LCST by volume-mean intensity, the LCST would be ˜155° C. In either case, the LCST for polyMSEA is far above 100° C., indicating that such neutral water-soluble polymers would not have solubility change issues when applied in various bio-related applications.

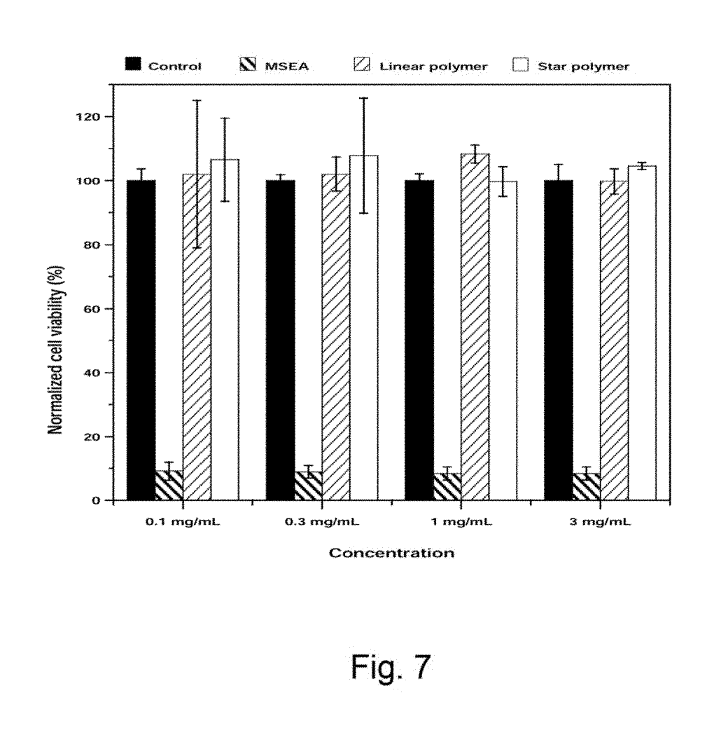

As described above, neutral biocompatible water-soluble polymers could represent a very significant class of polymeric materials that could provide high potential utility in various bio-medical applications. However, there are currently few polymers, synthesized by RDRP procedures, which fall into this category. One of these is polyOEGMA, which unfortunately is not very useful for a number of applications because of the high steric hindrance associated with the oligomeric side chain. As disclosed herein both linear polyMSEAs (Mₙ: ˜10,000) and star-like (for example, (polyMSEA)ₙ-polyDVB), which include a small MSEA repeating unit, were analyzed by cytotoxicity tests with human embryonic kidney cells (HEK293). As shown in FIG. 7, while the MSEA monomer was toxic, because of the presence of the acrylate groups, polyMSEA and (polyMSEA)ₙ-polyDVB were both non-toxic at concentrations up to, for example, 3 mg/mL.

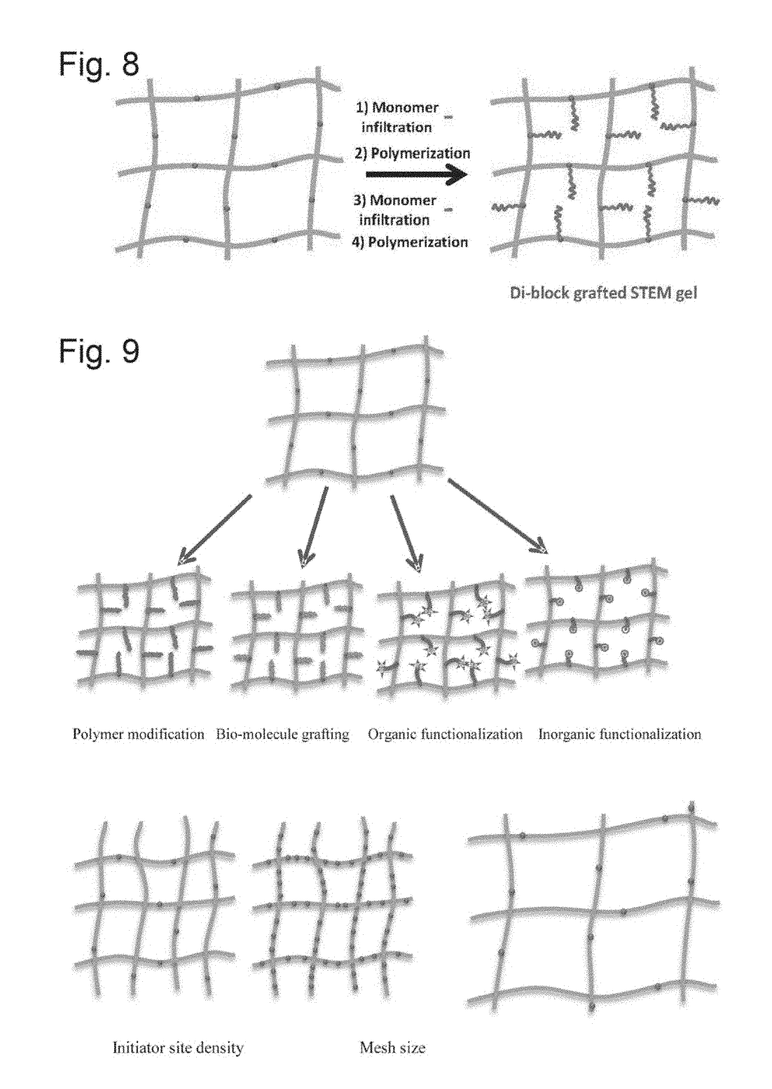

A polymer architecture that provides a suitable format for formation of bulk materials with controllable architecture, including controlled morphology may, for example, be based on a material we have identified as a structurally tailored and engineered macromolecular (STEM) gel. The procedure employed for a specific functionalization, with segmented copolymers, is shown in FIG. 8. The ability of the initial STEM gel to be functionalized by multiple compositionally different tethered molecules, which is shown in two dimensional schematic in FIG. 9, even though the gel is a regular three dimensional molecule, includes polymer modification, bio-molecule grafting, organic functionalization and inorganic functionalization in addition to different numbers of functional units along each link and different mesh size in the precursor functionalized STEM gel. The first formed STEM gel, in FIG. 9 is shown with a single functional group in each linear copolymer linkage of the gel structure, which is shown for the sake of clarity in the upper schematics. One or more functionals group can be incorporated depending on the targeted application. Moreover, the size/dimensions of the mesh may be also adjusted.

Of particular interest in the development of products based on biocompatible water-soluble sulfoxide functionalized monomers is linkage of these monomers to the STEM network to provide solid films with, for example, organized exemplary PMSEA channels which can be fabricated by preparing tethered chains with distributed sulfoxide units. The functional STEM gels can be prepared by infusing the first multifunctional STEM gel with a solution comprising a one or more monomers and conducting an RDRP/ATRP procedure to the desired degree of polymerization (DP). One may then slowly compress the gel to squeeze out, for example, at least 50% of the remaining monomer, preferentially more than 75%, and optimally at least 90% before replacing the solvent monomer mixture with a mixture of the monomer selected for the second grafted polymer segment and conducting a second grafting from reaction. One thereby forms, after removal of all remaining monomers and solvents, a flexible film that can act as an agent for site selective transdermal drug delivery.

Short interfering RNA (siRNA), together with plasmid DNA, has emerged as promising therapeutic agents for different kinds of diseases through RNA interference (RNAi). Examples of RNAi treatment include cancer, malaria, asthma, influenza, hepatitis and AIDS. The biological foundation of RNAi is based on siRNA's selective degrading property towards messenger ribonucleic acid (mRNA) to depress the gene expression. However, negatively charged “naked” siRNA will subject to electrostatic repulsion from also negatively charged plasma membrane. Moreover, the susceptibility to degradation of siRNA will prevent the siRNA from approaching the target cells.

Generally, there are two main types of siRNA carriers for gene delivery, i.e., viral delivery and nonviral delivery system. Viral delivery agents have high transfer efficiency, but are costly to produce, toxic to cells and may interfere with host genomes. Comparatively, nonviral counterparts are cheaper, more convenient to synthesize, most importantly can reveal various formats in structures to be applied in different circumstances. RDRP techniques, and particularly ATRP, potentially provide major contribution to the diversity of nonviral systems.

Cationic polymers offer high binding affinity for nucleic acids such as DNA and RNA and therefore could be used to form a polyplex. A further step to enhance bio-stability is a procedure known as PEGylation wherein polyethylene glycol are included with cationic polymers which allows the polyplex to be used as efficient siRNA delivery system. Previously, PEGylated star-like cationic polymers prepared via an arm-first approach have been reported for siRNA delivery.

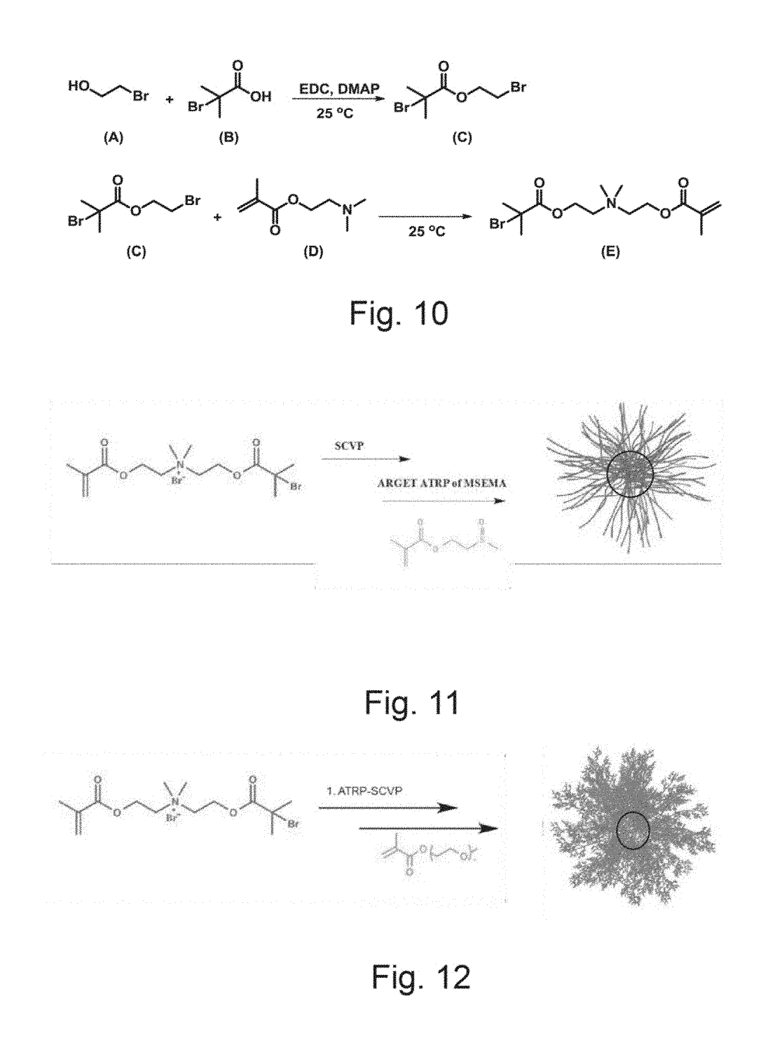

In another representative example hereof. AGET ATRP-based self-condensing vinyl polymerization (SCVP) was utilized to develop a new type of cationic hyperbranched polymer for binding siRNA. The procedure used for the preparation of the inimer employed as a comonomers in this SCVP procedure is shown below in FIG. 10.

By tuning the ratio of activator to deactivator, the degree of branching could be efficiently adjusted by moderating the number of monomer units added during each activation cycle. The positively charged hyperbranched molecules were conjugated with RNA, and, at concentrations lower than 12 μg/ml, resulted in survival % vs. control of over 90%. Biocompatibility may, for example, be improved by grafting a biocompatible shell from the cationic core. Two types of polymer shell were used to obtain biocompatibility, polyOEGMA and polyDMSO. From Agarose gel experiments, it was determined that the polyDMSO shell copolymer illustrated in FIG. 11 has a different influence on the binding efficiency as a result of relative low steric hindrance compared to the topology of the brush-like polyOEGMA copolymer of FIG. 12. In each of FIGS. 11 and 12, the polymer core is represented schematically with a circle.

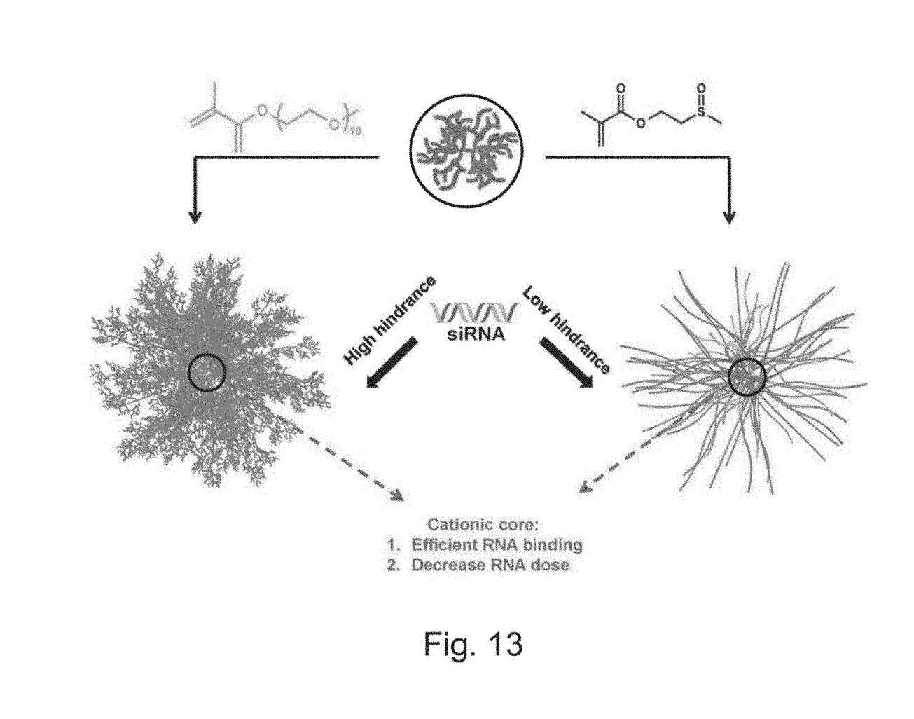

A series of Agarose gel experiments for the hyperbranched polymers were carried out. The sample with the polyOEGMA shell behaved in a manner equivalent to PEGylation, displaying high steric hindrance and hence lower binding and releasing efficiency. The sample with a pDMSO shell showed low steric hindrance, higher siRNA binding efficiency and also higher releasing efficiency. FIG. 13 illustrates the synthesis of the two hyperbranched polymers and incorporation of the representative nucleic acid siRNA.

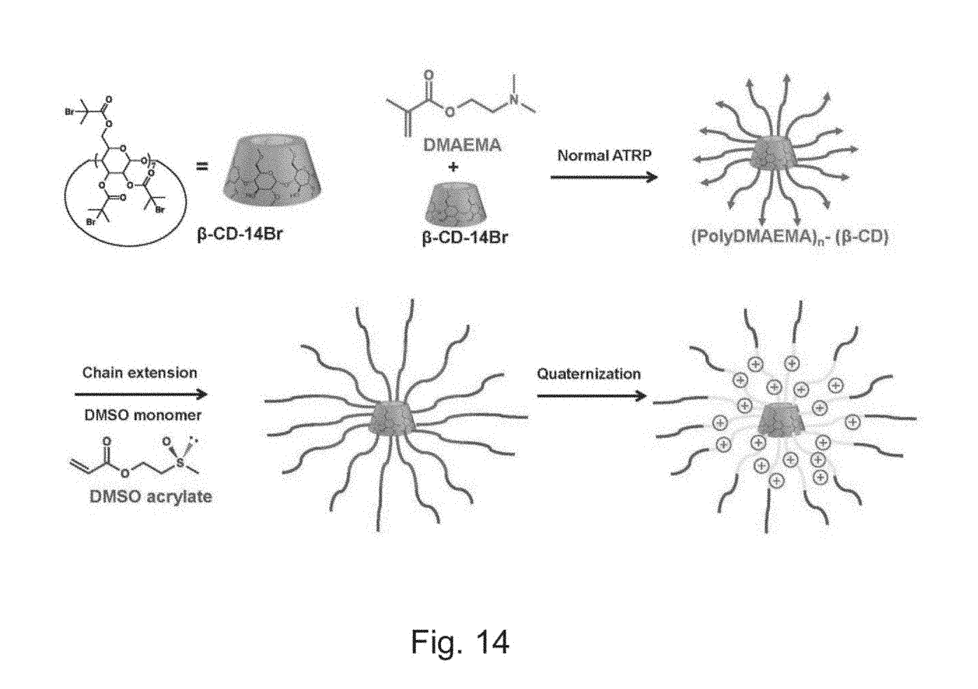

In addition to evaluating the utility of hyperbranched core functionalized core/shell molecules it was also of interest to make core-shell structures with different topological cores. β-cyclodextin (β-CD) was selected as a representative core forming molecule as the core itself could be used for transportation of small molecule drugs. Therefore, it was also of interest to use a multifunctional β-CD (14 Br per molecules) as a core (see FIG. 14). β-Cyclodextrin (β-CD) is a water soluble biocompatible, biodegradable, multifunctional molecule. Moreover, the hydrophobic cavity of cyclodextrin and its unique host-guest effect make it a good platform for hydrophobic drug delivery. To prepare a star polymer with β-CD as a core, a β-CD ATRP macroinitiator with 14 attached bromoisobutyrate sites was synthesized by esterification of accessible hydroxyl groups. The multi-arm star copolymer has a comparatively more uniform structure than the core/shell copolymer based on the hyperbranched cores discussed above.

The β-CD macroinitiator could be degraded in 5 wt % NaOH aqueous solution, 10 mg/mL, overnight. After degradation, the molecular weight of the initial sample at ˜3,000 shifted to oligomer peaks at ˜270. To demonstrate the biodegradability of the as synthesized star polymer (polyMSEA)ₙ-(β-CD), a star shaped copolymer was prepared under the same conditions employed for formation of a linear polymer with conversion of 26.8%, M_{n,GPC}=23,800 and M_{w}/Mₙ=1.05. The star copolymer sample was treated with 5 wt % NaOH aqueous solution overnight (10 mg/mL). After degradation, the original star peak shifted to M_{n,GPC}=2,300 with dispersity M_{w}/Mₙ=1.03, a size small enough for removal from the bloodstream. Therefore, such a high molecular weight biodegradable star-like structure, with low molecular weight side-arms, is suitable drug delivery applications, especially for delivery of small hydrophobic drugs.

In one example MSEA was grafted directly from the macroinitiator in a “core-first” manner by ARGET ATRP. The molecular weight increased regularly with conversion and the GPC traces moved cleanly to higher molecular weight. The final polymers displayed M_{n,GPC}=29,240 (M_{w}/Mₙ=1.06) for (polyMSEA)ₙ-(β-CD) at conversion of 38.5% measured by GPC with PMMA standards and M_{n,LS}=46,300 by static light scattering method (M_{w}/Mₙ 1.09).

Transdermal studies conducted with linear polyDMSO and cyclodextrin based star polyDMSO samples with molecular weight (5K, 10K, 50K, 100K) for each were prepared to compare the influence of morphology. The studies were carried out in a Franz cell from Perme Gear. The delivery from both receptor chamber as well as from the skin by doing kinetic study were examined in the presence of Rhomamine B methacrylate will be included with fraction no lower than 1%. After the experiment with mouse works, artificial human skin from MatTek will be examined.

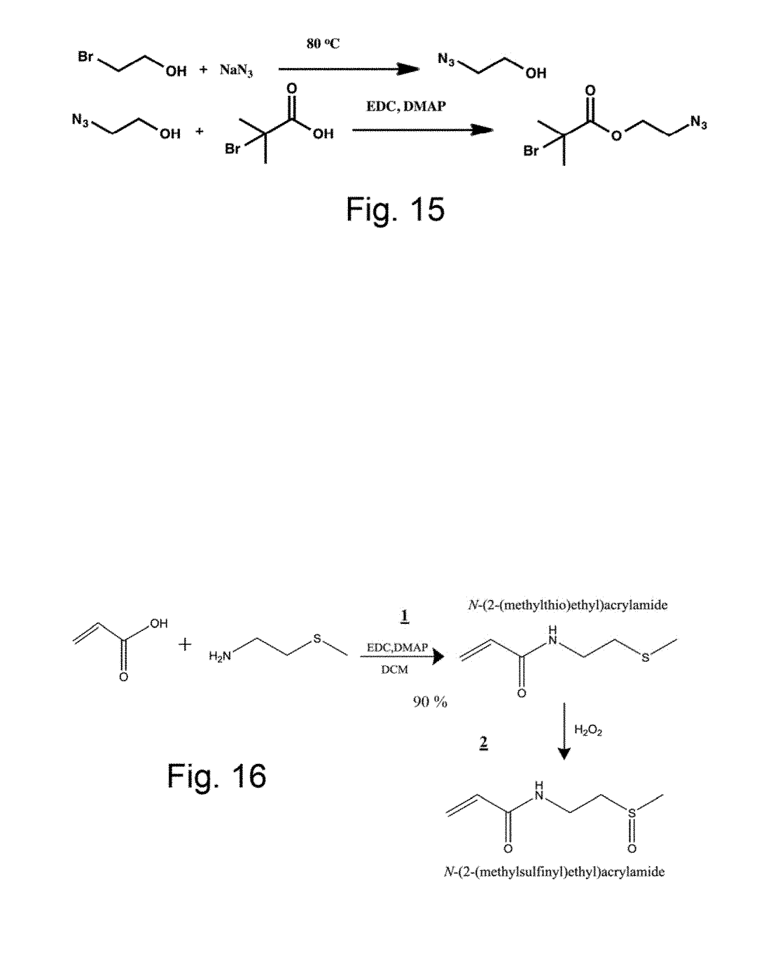

To further exemplify the utility of PDMSO modified materials for the preparation of bio-conjugated material for delivery applications an azide initiator, N₃-EBiB, was synthesized, as shown below in FIG. 15. Subsequently, DMSO acrylate was grown from the initiator by AGET ATRP to form an azide terminated polyDMSO.

The azide functionality can, for example, be used to tether the polymer to bio-molecules with suitable alkynyl functionality in a reaction known as “click” functionalization. [Macromol. 2004, 37, 9308] An example of click functionalization is the formation of DNA bottlebrush hybrids where DNA is clicked to an azide terminated brush copolymer thereby allowing formation of hundreds of duplex DNA strands that can accommodate hundreds of covalently attached and/or thousands of noncovalently intercalated fluorescent dyes can detect protein targets in flow cytometry, confocal fluorescence microscopy, and dot blots with an exceptionally bright signal. The formation of bottlebrush hybrids with polymers other than the sulfoxide-containing polymers hereof is disclosed in U.S. patent application Ser. No. 15/802,066, the disclosure of which is incorporated herein by reference. Sulfoxide containing polymers or PDMSO polymers hereof can now be linked to bio-molecules using the same click linkage procedure.

The synthesis of DMSO-Acrylamide monomer, as, for example illustrated in FIG. 16, was carried out to prepare a biocompatible polymer without an ester group in its structure. The DMSO-acrylamide monomer was used to synthesize a polymer on the surface of lipase TL. The absence of an ester group on the monomer, makes it resistant to the catalytic activity of Lipase. Indeed, the monomer is more stable against hydrolysis and more water-soluble, in addition to exhibiting lower cytotoxicity for the polymer.

The ¹H NMR spectrum of N-(2-(methylthio)ethyl)acrylamide showed that the monomer was prepared with an initial purity of 85%. While the oxidation and yield for monomer with two or three carbons were perfect, it was difficult to completely remove the residual H₂O₂ from the monomer. The residual hydrogen peroxide quickly polymerized the synthesized monomer even when they are diluted in methanol and stored in the freezer. It was determined that it was possible to remove the H₂O₂ by washing the aqueous solution with 20 times higher volume of DCM. The synthesized three carbon acrylamide monomer was water soluble. Indeed, it is even more water soluble than the acrylate version.

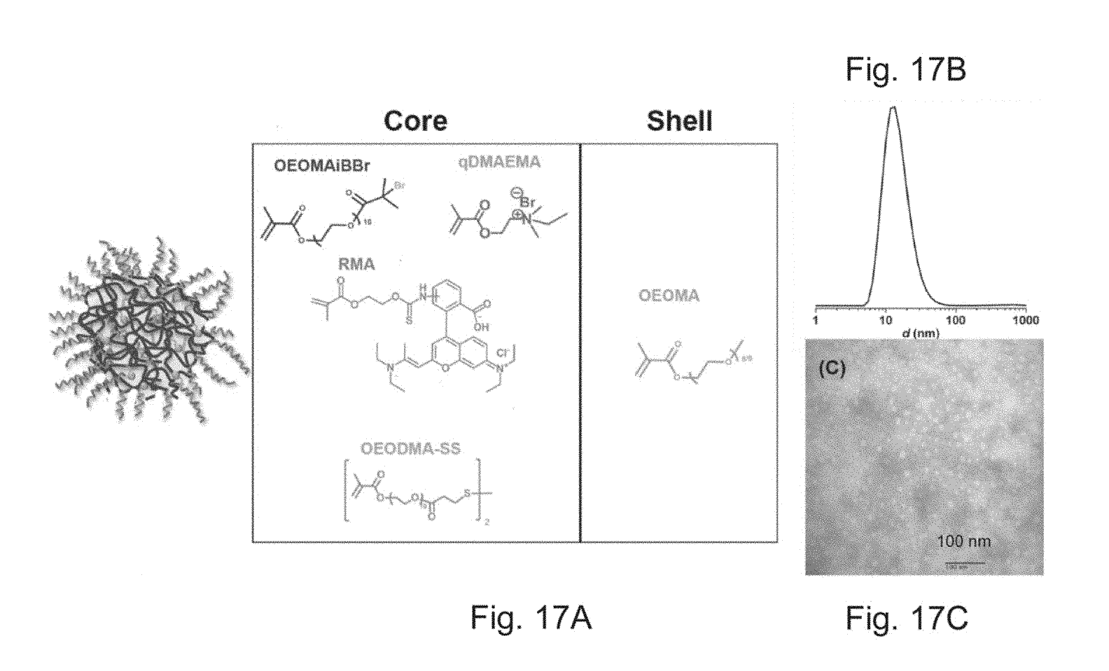

Another class of polymer based agents for siRNA delivery are known as nanogels. [Biomacromolecules 2012, 13, 3445] Functional cationic nanogels containing quaternized 2-(dimethylamino)ethyl methacrylate and a cross-linker with reducible disulfide moieties (qNG) were prepared by activators generated by electron transfer (AGET) atom transfer radical polymerization (ATRP) in an inverse miniemulsion. FIGS. 17A through 17C illustrate schematically the reparation and characterization of cationic core-shell nanogels which, may, for example, be used for DNA or RNA (for example, siRNA) delivery. In that regard, FIG. 17A illustrates the reagent added to the precursor of the nanogel and composition of the grafted-from shell. FIG. 17B illustrates the size distribution, and FIG. 17C illustrates a transmission electron microscopy of the particles. Literature (see, for example, Biomacromolecules 2014, 15, 4111; Chem. Rev. 2008, 108, 3747) suggests that smaller particles, of a size below 100 nm, are more efficient for the delivery of nucleic acids, because of formation of polyplexes that can undergo endosomal escape from the smaller particles. Therefore, nanogels particles were synthetized by ATRP in inverse microemulsion, with droplets size of ca. 20 nm. This procedure avoids Ostwald ripening, which resulted in a small and well-defined size of the obtained nanogels (diameter ca. 15 nm, FIG. 17B-17C). The nanogels are biocompatible and biodegradable. The presence of a labile crosslinker such as a disulfide crosslinker allows for the degradation under reducing environment. Preliminary biological evaluation showed that nanogels were biocompatible within measured range up to 0.2 mg/ml. The cationic charge within the core of the particles facilitate binding of siRNA. To optimize the binding conditions, four nanogels were prepared with different amount of cationic monomer in the core (0.5% 2, 4, prepared (pOEOMA, p(sulfobetaine-MA), and p(DMSO-MA).

Thus, in a number of embodiments of compositions hereof, building upon the analogous structure and utility of DMSO, well-defined stable polymeric homologues of DMSO were synthesized based on a neutral-charged water-soluble sulfoxide-containing monomer, 2-(methylsulfinyl)ethyl acrylate (MSEA). Well-defined linear polyMSEA was synthesized with low dispersity (<1.3) by ARGET ATRP utilizing low amounts of copper (100 ppm) based catalyst. Other ATRP procedures including photoATRP, and metal-freeATRP could also be employed. Furthermore, mono-dispersed star-like copolymers were synthesized as, for example, potential drug carriers by ARGET ATRP of 2-(methylthio)ethyl acrylate (MTEA) with post-oxidation of the arm precursor macroinitiator/macromonomer or the first-formed star macromolecule. In another representative embodiment, DMSO modeled functional units hereof were distributed in a robust form of a structurally tailored and engineered macromolecular (STEM) gel prepared. Such STEM gels may, for example, be used for DNA, RNA and/or transdermal drug delivery.

### EXPERIMENTAL EXAMPLES

Materials.

2-(methylthio)ethanol (≥99%) was purchased from Alfa Aesar. Hydrogen peroxide solution (30%) was purchased from Fisher Scientific. Acrylic acid (299%), tin(II) 2-ethylhexanoate (Sn^{II}(EH)₂, ≥95%), methyl acrylate (≥99%), n-butyl acrylate (≥99), poly(ethylene glycol) methyl ether acrylate (OEGA, average Mₙ=500), N-(3-Dimethylaminopropyl)-N′-ethylcarbodiimide hydrochloride (EDC, ≥99%), N,N-dimethylamino pyridine (DMAP, 2991%), copper bromide (≥99.99%), ascorbic acid, (AA, ≥99%), divinyl benzene (DVB, technical grade, 80%), ethyl α-bromoisobutyrate (EBiB, 98%) were purchased from Sigma Aldrich. Acrylic acid was distilled under vacuum before use. All acrylate monomers were passed through basic alumina columns before use. Tris(2-pyridylmethyl)amine (TPMA) was synthesized according to previous procedures. All solvents and other chemicals are of reagent quality and were used as received unless special explanation.

Instrumentation.

¹H nuclear magnetic resonance (NMR) measurements were performed on a Bruker Avance 300 MHz spectrometer. Molecular weight and molecular weight distribution (M_{w}/Mₙ) were determined by combination of gel permeation chromatography and multi-angle light scattering (GPC-MALS). The GPC-MALS used a Waters 515 HPLC pump, Wyatt Optilab refractive index detector, Wyatt DAWN HELEOS-II multi-angle light scattering detector and PSS columns (Styrogel 10², 10³, and 10⁵ Å) with either 40° C. THF or 50° C. DMF (50 mM LiBr) solution as eluent phase at flow rate of 1 mL/min.

### Example 1 Preparation of Linear Polymers. 1A) Synthesis of 2-(Methylsulfinyl)Ethyl Acrylate (MSEA)

2-(methylthio)ethyl acrylate (MTEA) was synthesized according to our previous work in a typical oxidation procedure. 5 g MTEA (1 equiv.) was added to a 25 mL round bottom flask sealed with rubber stopper. The flask was kept in an ice bath and purged by N₂. 3.77 g hydrogen peroxide solution (1.1 equiv.) was slowly injected into the flask at rate of 50 μL/min. The reaction was allowed to stir for 24 h and stopped by adding 50 mL deionized water. The aqueous solution was washed 3 times with 100 mL dichloromethane. The organic phase was collected and dried over magnesium sulfate. Excess solvent was removed under vacuum to give 4.5 g pure product of MSEA (yield 81.2° %). ¹H NMR (300 MHz, DMSO-d₆): δ (ppm)=6.38 (1H, dd, CHH═H), 6.19 (1H, q, CHH═H), 5.98 (1H, dd, CHH═H), 4.49 (2H, m, C═OOCH₂CH₂), 3.15 (1H, m, CH₂CHHS═O), 2.99 (1H, m, CH₂CHHS═O), 2.60 (3H, s, S═OCH₃).

1B) Synthesis of Linear polyMSEAs.

The polymerization of MSEA was carried out using ARGET ATRP. In a typical procedure, 8.6 mg EBiB (1 equiv.), 0.5 g MTEA (70 equiv.) 0.69 mg copper (II) bromide (0.07 equiv.), 1.9 mg TPMA (0.15 equiv.), 0.1 mL DMF and 2.9 mL DMSO were mixed in a sealed 10 mL Schlenk flask equipped with a stirring bar. The Schlenk flask was degassed by three freeze-pump-thaw cycles. The flask was allowed to warm up to room temperature and placed in an oil bath thermostated at 35° C. Deoxygenated ascorbic acid (AA) solution (5.4 mg in 0.1 mL DMSO) was injected into the flask under N₂ purge to trigger the polymerization, and an initial sample (t=0) was collected by syringe. Samples were taken periodically to measure conversion via ¹H NMR and molecular weight via GPC. The polymer was precipitated by adding the solution to ether and purified by dialysis (MWCO=100˜500 Da) against water, and dried by lyophilization.

1C) Measurement of Reactivity Ratios.

The reactivity ratio measurements were conducted according to the Kelen-Tüdös approach. [J. Polym. Sci.: Polym. Letters Ed. 1976, 14, 513, the disclosure of which is incorporated herein by reference] For example for the MSEA/MA pair four ARGET ATRP reactions with monomer 1: MSEA and monomer 2: MA, were conducted with the ratio of reagents [MSEA]₀/[MA]₀/[EBiB]₀/[AA]₀/[Cu₂Br]₀/[TPMA]₀=x/(70−x)/1/0.7/0.07/0.15 in DMSO at the same concentration as the above section. (x=60, 45, 25, 10). For example of MSEA/nBA pair (monomer 1: MSEA, monomer 2: nBA), four ARGET ATRP reactions were conducted with a ratio of reagents [MSEA]₀/[nBA]₀/[EBiB]₀/[AA]₀/[Cu₂Br]₀/[TPMA]₀=x/(70−x)/1/0.7/0.07/0.15 in DMSO at the same concentration as the above section (x=60, 45, 35, 10). For the MSEA/OEGA pair (monomer 1: MSEA, monomer 2: OEGA), four ARGET ATRP reactions were conducted at condition of [MSEA]₀/[nBA]₀/[EBiB]₀/[AA]₀/[Cu₂Br]₀/[TPMA]₀=x/(70−x)/1/0.7/0.07/0.15 in DMSO at the same concentration as the above section (x=60, 45, 35, 10). The conversion of monomers was measured by ¹H NMR. All reactions were stopped within 15 min to ensure a low degree of conversion.

### Example 2 Synthesis of Star-Like Polymers. 2A) Preparation of a Linear Macroinitiator

Star synthesis was accomplished using a post-oxidation approach. First, linear polyMSEA macroinitiators were synthesized by ARGET ATRP. In a typical procedure, 0.222 g EBiB (1 equiv.), 10 g MTEA (60 equiv.) 17.8 mg CuBr₂ (0.07 equiv.), 49.7 mg TPMA (0.15 equiv.) were mixed in a solution of 11 mL acetone and 0.5 mL DMF in a sealed 50 mL Schlenk flask equipped with a stirring bar. The Schlenk flask was degassed by three freeze-pump-thaw cycles. The flask was allowed to warm up to room temperature and placed in an oil bath thermostated at 35° C. Deoxygenated ascorbic acid (AA) solution (140 mg in 0.5 mL DMF) was injected into the flask under N₂ purge to activate the polymerization, and an initial sample (t=0) was collected by syringe. Samples were taken periodically to measure conversion via ¹H NMR and molecular weight via GPC. The polymer, with Mₙ of 3000, was precipitated by addition of the reaction solution to methanol and the filtered solids were dried under vacuum.

2B) Formation of a Star.

The polysulfide star was synthesized by ARGET ATRP using polyMTEA as macroinitiator in a chain extension polymerization with a divinyl monomer. In a typical procedure, 470 mg polyMTEA (1 equiv.), 222 mg DVB (14 equiv.), 0.16 mg CuBr₂ (0.01 equiv.) and 3.5 mg TPMA (0.1 equiv.) were mixed in 4.5 mL anisole and 0.1 mL DMF in a sealed 10 mL Schlenk flask equipped with a stirring bar. The Schlenk flask was degassed by three freeze-pump-thaw cycles. The flask was allowed to warm up to room temperature and placed in an oil bath thermostated at 90° C. A deoxygenated Sn(EH)₂ solution (9.9 mg in 0.5 mL anisole) was injected into the flask under N₂ purge to reduce the cupric complex and trigger the polymerization, and an initial sample (t=0) was collected by syringe. Samples were taken periodically to measure conversion via ¹H NMR and molecular weight via GPC-MALS. The final polymer was precipitated in ether to remove non-polymerized DVB and purified by dialysis (MWCO=10,000 Da) against THF and dried under vacuum. The polysulfoxide star, (polyMSEA)ₙ-polyDVB, was synthesized by post-oxidation. In a typical procedure, 0.1514 g (polyMTEA)ₙ-polyDVB (1 equiv. per MSEA repeating unit) was dissolved in 1 mL deoxygenated THF in a sealed 10 mL round-bottom flask equipped with a stirring bar. 121.3 mg hydrogen peroxide solution (1.3 equiv.) was slowly injected at a rate of 50 μL/min and allowed to react for 72 h. After the reaction was finished, the solution was precipitated by addition into 10 mL THF and purified by dialysis (MWCO=10,000 Da) against deionized water and dried under vacuum to give pure product of (polyMSEA)ₙ-polyDVB. The molecular weight of the star polymers was analyzed by GPC-MALS.

### Example 3. Samples for Evaluation of LCST

The polymers for LCST evaluation were prepared by copolymerizing different ratios MSEA with N-isoprypylacrylamide (NIPAM) by free-radical polymerization (FRP). In a typical procedure, 100 mg MSEA (12 equiv.), 104.7 mg NIPAM (18 equiv.) and 8.53 mg AIBN (1 equiv.) were dissolved in 2 mL DMF in a 10 mL Schlenk flask equipped with a stirring bar. The flask was sealed and degassed by three freeze-pump-thaw cycles. The flask was allowed to warm up to room temperature and placed in an oil bath thermostated at 70° C. All reactions were allowed to go progress to >99% conversion and precipitated into ether. The final product was collected and dried under vacuum oven overnight. For DLS analysis, 30 mg of the as-dried copolymer was dissolved in 3 mL deionized water and passed through a 0.2 micron PTFE filter.

### Example 4. Cytotoxicity Test

Human Embryonic Kidney Cells (HEK 293) were grown in a 96 well tissue culture plate at a density of 10,000 cells/well for 24 hours. A solution of polyMSEA and starMSEA polymers (100 mg/mL) was prepared by mixing with 1×PBS and then added to the tissue culture plate at concentrations of 10 μg, 30μ, 100 μg, and 300 μg. All concentrations were tested in triplicate. The polymer was then incubated with the HEK 293 cells for 48 hours at 37° C. in the presence of 5% CO₂. After 48 hours, ATP Cell Titer Glo® Assay was used and luminescence was measured and quantitated as a percentage of the control wells.

### Example 5. Functional STEM Gels

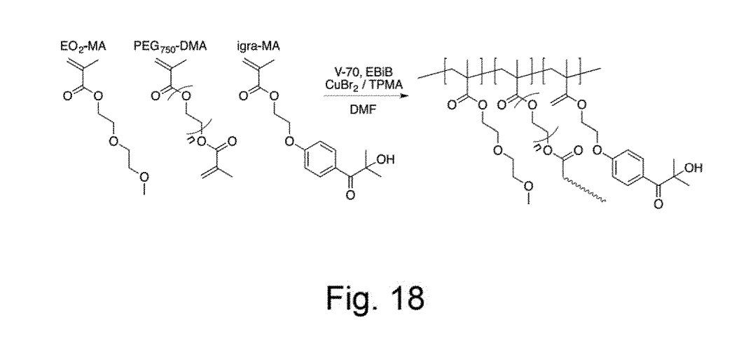

The formation of different STEM gels, identified as ATRP 75, ATRP 150, ATRP 300 according to their different mesh sizes, which directly depend on the [M]:[I], ratio, were synthesized under the following conditions ([E02-MA]0: [PEG750-MA]0: [irga-MA]0: [EBiB]0: [V-70]0: [CuBr₂]0:[TPMA]0=[(75,150,300)]:[5]:[1]:[1]:[0.25]:[0.1]:[0.4] in DMF at 45° C. (EO₂-MA: Di(ethylene glycol)methyl ether methacrylate, PEO₇₅₀-DMA: Poly(ethylene glycol)dimethacrylate, average MW=750, irga-MA: 2-(4-(2-hydroxy-2-methylpropanoyl)phenoxy)ethyl methacrylate) (see FIG. 18).

The swelling ratio of the gels in water were measured in order to determine if the different mesh sizes resulted in different swelling ratio's. The results are listed in Table 1 below and indicate that gels with different mesh sizes were prepared.

TABLE 1

| | | | Irga | | Swelling |
|---|---|---|---|---|---|
| | [EO₂-MA]:[PEO₇₅₀- | Mesh | ratio | | ratio |
| Exp. | DMA]:[irga-MA] | size^{a} | (mol %)^{b} | Conv.^{c} | (24 h)^{d} |
| 1-1 | 75:5:1 (made by FRP) | 15 | 1.33% | — | 242% |
| 2-31 | 75:5:1 | 15 | 1.33% | — | 331% |
| 2-32 | 150:5:1 | 30 | 0.67% | — | 387% |
| 2-36 | 150:5:2 | 30 | 1.33% | 89% | 448% |
| 2-33 | 300:5:1 | 60 | 0.33% | — | 645% |
| 2-37 | 300:5:4 | 60 | 1.33% | 82% | 625% |

^{a}monomer divided by cross-linker ([EO₂-MA]/[PEO₇₅₀-DMA])
^{b}irga divided by monomer ([irga-MA]/[EO₂-MA])
^{c}Conversion was determined by NMR
^{d}swelled solvent weight (g)/original gel weight(g)

### Example 6. Synthesis and Properties of Hyperbranched Polymers and Core-Shell Structures. 6A) Synthesis of Hyperbranched Polymer and Core-Shell Structures

The synthesis of the inimer is described in FIG. 10. AGET ATRP was adopted for the preparation of the hyperbranched polymer. Three different ratio's of the reagents were used to synthesize the cationic hyperbranched cores.

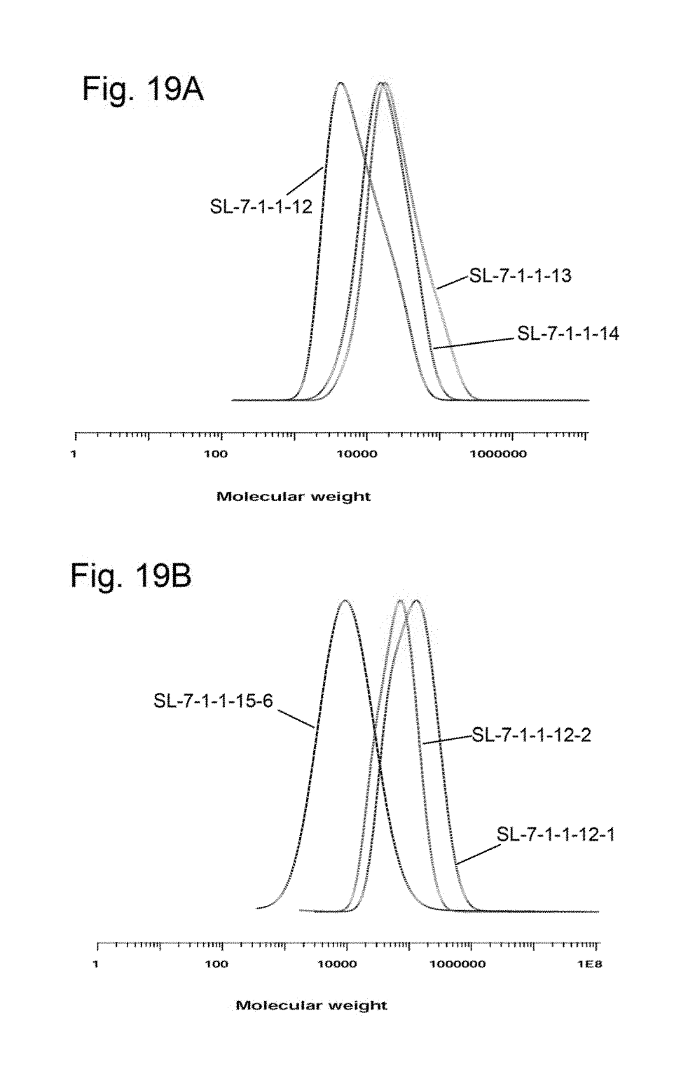

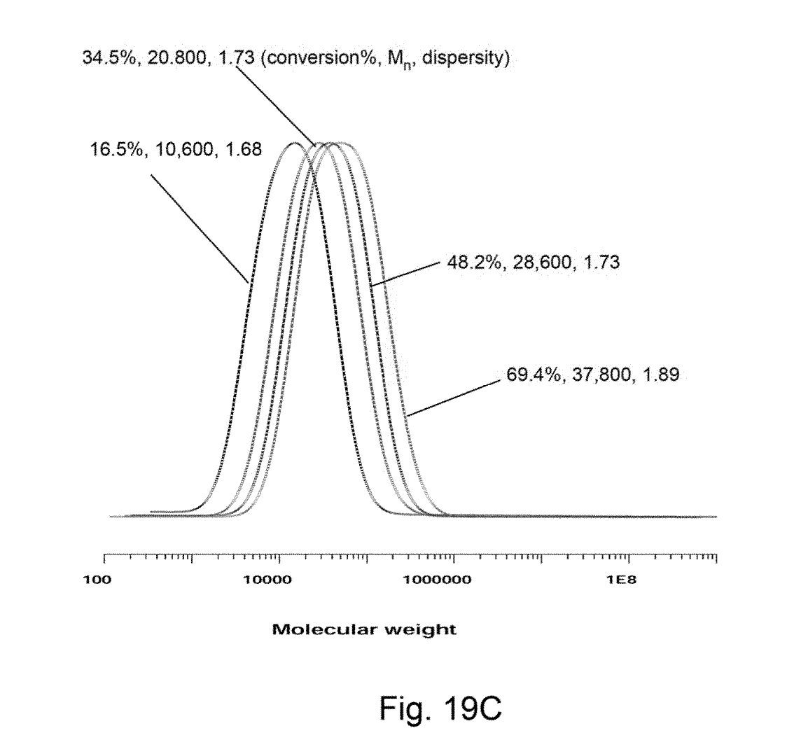

SL-7-1-1-12: [Inimer]/[AA]/[CuBr₂]/[TPMA]=50/0.3/4/6 (35° C. in DMSO m/m=5/8)
SL-7-1-1-13: [Inimer]/[AA]/[CuBr₂]/[TPMA]=50/1.3/4/6 (35° C. in DMSO m/m=5/8)
SL-7-1-1-14: [Inimer]/[AA]/[CuBr₂]/[TPMA]=50/4.4/4/6 (35° C. in DMSO m/m=5/8)
Table 2 below sets forth the macromolecular properties of different cationic hyperbranched polymer sample. GPC traces are illustrated in FIG. 19A.

TABLE 2

| Reaction # | Media | Cu^{I}/(Cu^{II} + Cu^{I}) | Conversion | M_{n(GPC)} | M_{n(NMR)} | Ð | DB |
|---|---|---|---|---|---|---|---|
| SL-7-1-1-12 | DMSO | 0.3/4 | 88.9% | 5,700 | 75,300 | 1.78 | 0.34 |
| SL-7-1-1-13 | DMSO | 1.3/4 | 92.6% | 19,000 | 60,100 | 1.81 | 0.22 |
| SL-7-1-1-14 | DMSO | 4/4 | 92.5% | 14,200 | 55,500 | 1.49 | 0.16 |

6B) PolyOEGMA shell was grown from core SL-7-1-1-12 with DB 0.34 by ARGET ATRP. The results are shown in Table 3 below and GPC traces are shown in FIG. 19B.

TABLE 3

| | | | | | Core- | Core- | Core- | Arms |
|---|---|---|---|---|---|---|---|---|
| | Core | Core | Core | Arm | Shell | Shell | Shell | per |
| Reaction # | M_{nGPC} | M_{nNMR} | DB | DP | Mₙ | Ð | Mₙ by LS | star |
| SL-7-1-1-15-7 | 5,700 | 78,300 | 0.34 | 5 | 8,500 | 1.37 | N/A | N/A |
| SL-7-1-1-12-2 | 5,700 | 78,300 | 0.34 | 48 | 69,300 | 1.21 | 47,600 | N/A |
| SL-7-1-1-12-1 | 5,700 | 78,300 | 0.34 | 95 | 127,000 | 1.33 | 1501,000 | 30 |

The structure with polyDMSO shells was synthesized by the following procedure: [MSEMA]/[one Br site]/[AA]/[CuBr₂]/[TPMA]=100/1/2/0.6/0.6, GPC traces of ARGET A TRP of MSEMA from cationic core with increasing conversion are shown in FIG. 19C.

6C) Biocompatibility, Zeta Potential and Agarose Gel Experiments.

The hyperbranched core sl-7-1-1-12 with DB of 0.34 was tested for biocompatibility with cell SH-SY5Y and compared with the biocompatibility tests of hyperbranched core with polyOEGMA shells. The pure cationic exhibited high toxicity and the PEGylated core shell structures showed high biocompatibility towards the SH-SY5Y shell.

The zeta potential of all core-shell structures were tested and shown in Table 4 below.

TABLE 4

| Sample | Shell DP | Zeta Potential (mV) |
|---|---|---|
| SL-7-1-1-12 | HB core, DB 0.34 | 38.1 |
| PolyOEGMA shell | | |
| SL-7-1-1-15-6 | 5 | 10.1 |
| SL-7-1-1-12-2 | 48 | 0.12 |
| SL-7-1-1-12-1 | 95 | 0.11 |
| PolyMSEA shell | | |
| SL-7-1-1-15-10 | 10 | 3.00 |
| SL-7-1-1-15-7 | 35 | 0.36 |

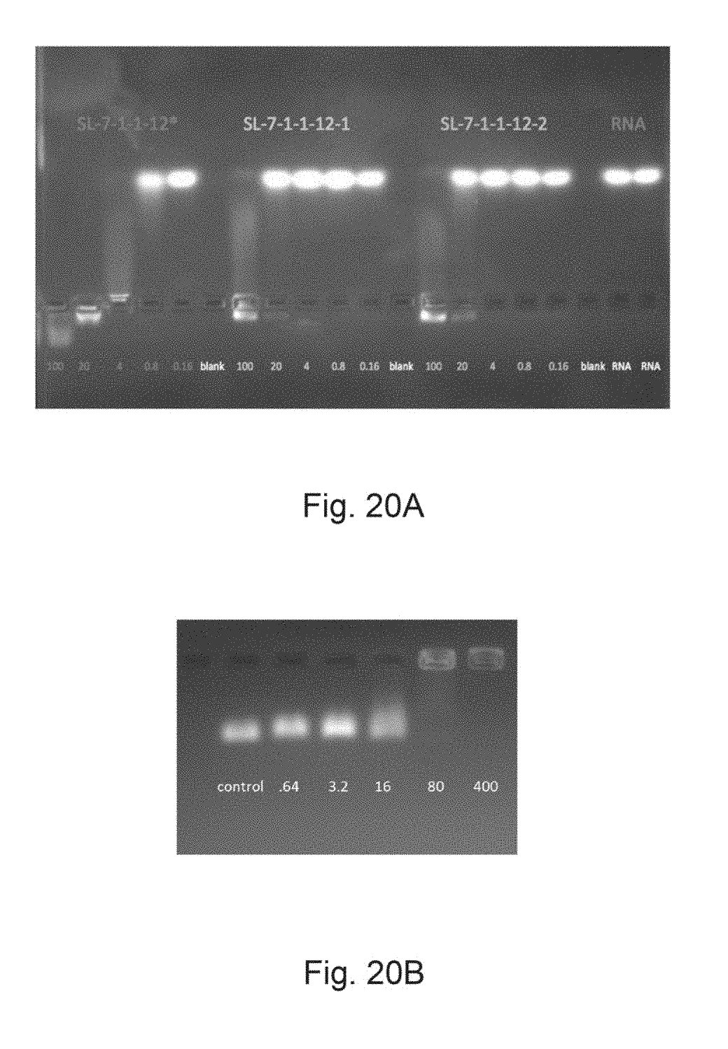

A comparison of the Agarose gel experiments of pure cationic core and the Agarose gel experiments of polyDMSO shelled samples with similar shell DP (see FIGS. 20A and 20B) shows that the core-shell structures with polyDMSO shell has higher binding efficiency. It started to bind at ratio of 16 and was nearly fully complexed at ratio of 80.

### Example 7 Cyclodextrin Based Star Copolymers

The schematic of the procedure used to prepare cyclodextrin based star copolymers is shown in FIG. 14.

7A) Initiator Functionalized β-Cyclodextrin:

10 g of β-cyclodextrin (β-CD, 8.81 mmol) was dried under vacuum overnight and placed in 250 mL round bottom flask. The β-CD was dissolved in 100 mL of anhydrous 1-methyl-2-pyrrolidone (NMP) and the flask was placed in an ice bath. 2-Bromoisobutyryl bromide (BriBBr) (27 mL, 1.2 eq. to —OH) was dissolved in anhydrous NMP (50 mL) and slowly added to the β-CD solution. The solution temperature was allowed to warm up to room temperature and the reaction was allowed to stir for 1 day. The dark brown solution was dialyzed against distilled water (MWCO=500) for 1 week. The remaining product was concentrated at reduced pressure and crystallized in cold hexane to obtain a pale yellow solid product.

7B) Formation of polyMTEA Star with β-CD Core.

The polyMTEA macroinitiators were synthesized by ARGET ATRP. In a typical procedure, 0.222 g EBiB (1 equiv.), 10 g MTEA (60 equiv.) 17.8 mg CuBr₂ (0.07 equiv.), 49.7 mg TPMA (0.15 equiv.) were mixed in a solution of 11 mL acetone and 0.5 mL DMF in a sealed 50 mL Schlenk flask equipped with a stirring bar. The Schlenk flask was degassed by three freeze-pump-thaw cycles. The flask was allowed to warm up to room temperature and placed in an oil bath thermostated at 35° C. Deoxygenated ascorbic acid (AA) solution (140 mg in 0.5 mL DMF) was injected into the flask under a N₂ purge to activate the polymerization, and an initial sample (t=0) was collected by syringe. Samples were taken periodically to measure conversion via ¹H NMR and molecular weight via GPC. The polymer with Mₙ=3000 was isolated by precipitation in methanol and dried under vacuum.

However, while the formation of a star copolymer with β-CD core was successful chain extension with DMAEMA was not successful.

7C) Formation of Poly(qDMAEMA) Star with β-CD Core.

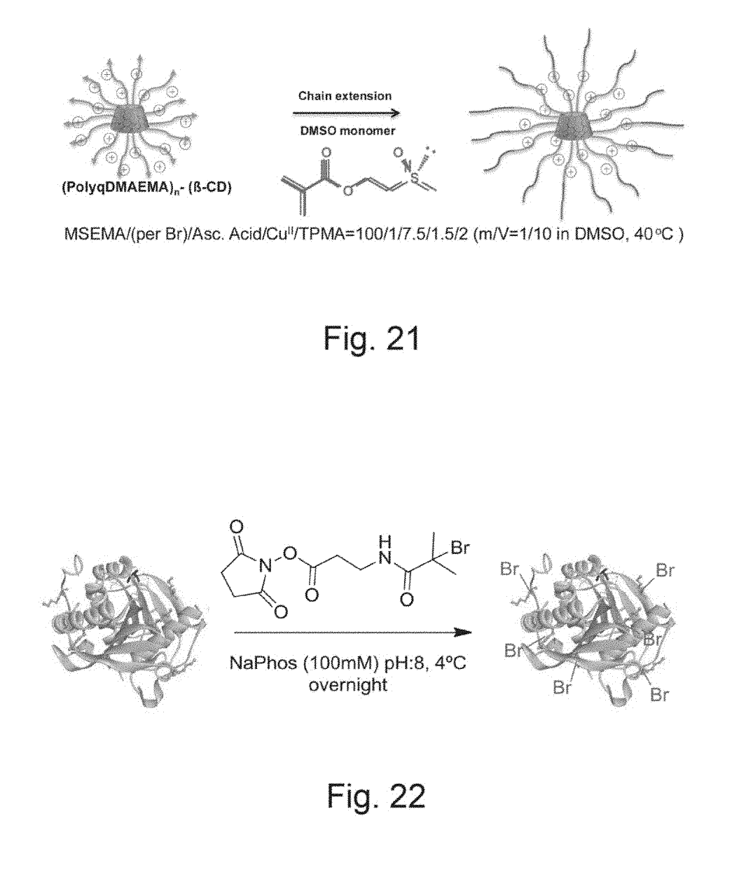

FIG. 21 illustrates schematically an alternative approach to forming a star delivery agent with segmented arms, in this case with a cation containing core. The star delivery agent may, for example, be prepared by initially grafting quaternized DMAEMA from the functionalized 3-CD using an ARGET ATRP procedure. The following initial ratio of reagents were used in a representative example: qDMAEMA/(per Br)/Asc. Acid/Cu^{II}/TPMA=70/1/3/0.14/0.5, (run SL-8-22-1). The second block was also carried out by a chain extension ARGET ATRP of DMSO methacrylate.

Samples with different length of each block were prepared. Interestingly, all the core shell polymers had very high surface potential as compared to the hyperbranched polymer based core shell polymers (comparable to PEI, which has a toxicity issue). This could mean that such cyclodextrin based polymers has higher performance at binding siRNA. The results are listed in Table 5 below:

TABLE 5

| | | 1^{st} | 1^{st} | 2^{nd} | | | | | | Core- |
|---|---|---|---|---|---|---|---|---|---|---|
| | | Block | Block | Block | | | | | | Shell |
| Entry # | Core | DP | Zeta (mV) | DP | M_{n, GPC} | Ð_{GPC} | M_{n, LS} | Ð_{LS} | M_{n, Theo} | Zeta (mV) |
| A | β-CD-Br₁₄ | 18 | N/A | 52 | 5300 | 1.29 | 10870 | 1.12 | 186100 | 41.3 |
| B | β-CD-Br₁₄ | 30 | 44.2 | 19 | 22600 | 1.48 | 68000 | 1.38 | 155400 | 37.0 |
| C | β-CD-Br₁₄ | 30 | 44.2 | 32 | 29200 | 1.60 | 105100 | 1.49 | 184510 | 32.5 |

### Example 8. Preparation of Grafted Biomolecules. 8A) Modification of Thermomyces Lanuginosus (TL) with Bromide Initiator

The protein (337 mg) in 100 mL of NaPhos buffer (pH:8 100 mM) was reacted with 20× excess of the fresh amide initiator (2,5-dioxopyrrolidin-1-yl 3-(2-bromo-2-methylpropanamido) propanoate) as illustrated in FIG. 22. The initiator was dissolved in 0.5 mL of DMSO and added dropwise to the solution. The reaction was carried out overnight at 4° C.

After the reaction was finished, the solution was purified by dialysis with a 25K membrane cut-off for 2 days. Then, the protein was frozen with liquid nitrogen and lyophilized for 48 hours in the lyophilizer. MALDI-TOF analysis comparison of the molecular weight of the native Thermomyces Lanuginosus, which corresponds to 29701 Da, with that of the substituted lipase which had a molecular weight of 30957 Da, corresponds to an initiator functionalization ratio of 5.7 substituted lysines with the initiator out of 6 accessible lysines. This experiment indicates that the full substitution of the 6 accessible lysines (7 total) occurred.

The amount of enzyme recovered after dialysis and lyophilization is close to 500 mg.

8B. Growth of PolyMSEA from Bovine Serum Albumin (BSA).

ARGET ATRP of MSEA from Bovine Serum Albumin (BSA-Br₂₇ MW=9840) was carried out under the following conditions: the ratio of reagents were [MSEA]/[one Br site]/[AA]/[CuBr₂]/[TPMA]/[NaBr]=100/1/0.7/0.14/0.5/30 (27 Br per BSA) (m/v=1/14 in H₂O, r.t.). The grafting from reaction progresses in a controlled manner over the initial 74% monomer conversion providing tethered polymer chains with a molecular weight of 126,500 and a Ð=1.4. Continued chain extension led to observable coupling in the GPC curves, due to the multiple growing chains on the BSA initiator and increased viscosity of the reaction medium.

### Example 9 Preparation of Clickable PDMSO

N₃-EBiB (wherein Ebib is ethyl-2-bromoisobutyrate) was used as the initiator to prepare a “clickable” PDMSO by growing DMSO acrylate from the initiator by AGET ATRP under the conditions in the following recipe: (run SL-8-21-1-1) M/N₃-EBiB/Asc. Acid/Cu^{II}/TPM=140/1/1.4/0.14/0.4 (m/V=1/6 in DMSO, 35° C.) after 17 h, the conversion reached 62.9% with a theoretical molecular weight of 14,500. The difference between theoretical and apparent molecular weight is a result of a difference of calibration. The dispersity could be lowered by stopping at lower conversion. The molecular weight is analyzed by DMF GPC. The molecular weight is 24,600 and dispersity is 1.8. This polymer was attached to proteins with complementary functionality.

### Example 10. Preparation and Characterization of Cationic Core-Shell Nanogels for siRNA Delivery. 10A) OEOMAiBBr Inimer Synthesis

The OEOMAiBBr inimer was synthesized via esterification. OEOMA_{500-OH} and was purified prior to use to remove PEGDMA. 2-Bromo-2-methylpropionic acid (0.10 mol, 22.10 g), and N-(3-dimethylaminopropyl)-N′-ethylcarbodiimide hydrochloride (0.15 mol, 27.90 g) were dissolved in 100 ml of DCM in a round bottom flask and cooled in an ice bath. OEOMA_{500-OH} (0.06 mol, 30 mL) and 4-(dimethylamino)pyridine (0.008 mol, 1.02 g) were dissolved in 100 ml of DCM and added dropwise into the solution in the reaction flask. The reaction flask was sealed and then the solution was removed from the ice bath and allowed to react overnight at room temperature. The inimer was purified by washing the solution with dilute HCl (0.1 M) three times, once with water, then three times with saturated NaHCO₃, followed by a final water wash. The final organic solution was then dried over magnesium sulfate, filtered and concentrated in a rotary evaporator. To prevent degradation and crosslinking inhibitor (hydroquinone) was added to the inimer solution prior to solvent removal. Final product was characterized by ¹H NMR.

10B). Synthesis of Cationic Core-Shell Nanogels Via AGET ATRP in an Inverse Microemulsion.

The nanogels were synthesized by inverse microemulsion AGET ATRP. The aqueous phase consisted of a solution of OEODMA (0.133 mmol, 100.0 mg), OEOMAiBBr (0.6 mmol, 400 mg), qDMAEMA (0.08 mmol, 20 mg) in 275 mg of Cu(II)Br₂/TPMA stock solution in water (1/1.1, 5.5 mg/ml of CuBr₂), 275 mg of ultrapure water and 0.1 mg of methacryloxyethyl thiocarbamoyl rhodamine B. Different amount of qDMAEMA were added in different batches. The aqueous phase and 1.5 g of surfactant were added to 11.5 ml of hexane. The solution was vigorously shaken to form a stable microemulsion. The solution was degassed by bubbling with nitrogen for 15 minutes, and 4 μl of hydrazine hydrate was injected to the reaction under nitrogen to initiate the polymerization. After 3 hours more 11.5 ml of hexane was added. Then solution of 500 mg of OEOMA₅₀₀ (or other monomer) in 275 mg of Cu(II)Br₂/TPMA stock solution in water (1/1.1, 5.5 mg/ml of CuBr₂) and 275 mg of ultrapure water was added. Mixture was stabilized by addition of 1 ml of surfactant. The solution was degassed by bubbling with nitrogen for 15 minutes, and 8 μl of hydrazine hydrate was injected to the reaction under nitrogen to initiate the polymerization. Reaction was allowed to run overnight and was stopped by exposing the microemulsion to air. The nanogels solution was diluted with 10 ml of water, and the mixture was poured in 300 ml of a solution containing a 3:1 ratio of hexane/l-butanol and stirred for 2 h. After that aqueous layer was dialyzed in the sequence of solvents THF-water-acetone-water-acetone-water 10 h each, and then 2 more times against water to obtain a final solution, which was stored in the fridge.

P(OEOMA), P(sulfobetaine-MA), and P(DMSO-MA) shells were grafted from the accessible initiators present on the surface of the nanogel particles using ARGET ATRP.

The effect of incorporating different ratio's of qDMAEMA within the core of the gel particle was measured and the results are presented in the Table 6 below.

TABLE 6

| % WT QDMAEMA | D (NM) | Z (MV) | CONC (MG/ML)* |
|---|---|---|---|
| 0.5 | 18 ± 3 | 6.6 ± 0.9 | 25.2 |
| 2 | 19 ± 5 | 16.2 ± 2.3 | 5.6 |
| 6 | 16 ± 6 | 27.2 ± 1.4 | 13.8 |
| 10 | 22 ± 3 | 28.1 ± 0.7 | 19.5 |
| 18* | 13 ± 3 | 25.1 ± 2.1 | 11.8 |

Electrophoresis was employed to measure complexation to siRNA an showed that complexation increased with higher levels of incorporated qDMAEMA.

The effect of different compositions on the shell on the siRNA complex formation was also measured for particles containing 6% qDMAEMA and are shown in the Table 7 below.

TABLE 7

| SHELL COMPOSITION | D (NM) | Z (MV) | CONC (MG/ML)* |
|---|---|---|---|
| P(OEOMA₅₀₀) | 16 ± 6 | 27.2 ± 1.4 | 13.8 |
| P(SULFOBETAINE-MA) | 23 ± 9 | 16.0 ± 1.6 | 14.4 |
| P(DMSO-MA) | 17 ± 4 | 18.6 ± 0.9 | 29.0 |

Nanoparticles with a P(DMSO-MA) shell were able to form a complex with a higher fraction of si RNA which could be attributable to steric effects.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A biocompatible, water-soluble polymer comprising segments formed via an atom transfer radical polymerization and comprising sulfoxide functionality wherein the segments comprising sulfoxide functionality have a dispersity less than or equal to 1.75.

2. The biocompatible, water-soluble polymer of claim 1 wherein the segments comprising sulfoxide functionality have a dispersity less than or equal to 1.3.

3. The biocompatible, water-soluble polymer of claim 1 wherein the segments comprising sulfoxide functionality are formed via an atom transfer radical polymerization of monomer including at least one of 2-(methylsulfinyl)ethyl acrylate or 2-(methylthio)ethyl acrylate.

4. The biocompatible polymer of claim 1 wherein the segments comprising sulfoxide functionality are linear polymer segments or comprise segments forming branched macromolecules, comb-shaped macromolecules, star macromolecules, nanoparticles, or segments in a gel structure.

5. The biocompatible, water-soluble polymer of claim 1 wherein the segments comprising sulfoxide functionality are formed by post-polymerization oxidation of a thio-functional group.

6. The biocompatible, water-soluble polymer of claim 1 wherein the segments comprising sulfoxide functionality are polymerized using an activator regeneration by electron transfer atom transfer radical polymerization process.

7. The biocompatible, water-soluble polymer of claim 1 wherein the polymer does not exhibit a LCST in aqueous solution below 100° C.

8. The biocompatible, water-soluble polymer of claim 1 wherein the polymer segments comprising sulfoxide functionality comprises one or more radically copolymerizable monomers other than sulfoxide-containing monomer or thio-containing monomers.

9. The biocompatible water-soluble polymer of claim 1 wherein the polymer is a linear polymer, a star shaped polymer, a core/shell macromolecule or a component of a gel.

10. The biocompatible water-soluble polymers of claim 7 wherein the polymers are attached to a natural biological molecule including DNA, RNA, a protein or an enzyme.

11. A method of forming a biocompatible, water-soluble polymer comprising forming segments thereof via an atom transfer radical polymerization which comprise sulfoxide functionality, wherein the segments comprising sulfoxide functionality has a dispersity less than or equal to 1.75.

12. The method of claim 11 wherein the segments comprising sulfoxide functionality have a dispersity less than or equal to 1.3.

13. The method of claim 11 wherein the segments comprising sulfoxide functionality are formed via an atom transfer radical polymerization of monomer including at least one of 2-(methylsulfinyl)ethyl acrylate or 2-(methylthio)ethyl acrylate.

14. The method of claim 11 wherein the segments comprising sulfoxide functionality are linear polymer segments or comprise segments forming branched macromolecules, comb-shaped macromolecules, star macromolecules, nanoparticles, or segments in a gel structure.

15. The method of claim 11 wherein the segments comprising sulfoxide functionality are formed by post-polymerization oxidation of a thio-functional group.

16. The method of claim 11 the segments comprising sulfoxide functionality are formed using an activator regeneration by electron transfer atom transfer radical polymerization process.

17. A carrier for delivery a molecule comprising a biocompatible, water-soluble polymer comprising segments formed via an atom transfer radical polymerization and comprising sulfoxide functionality wherein the segments comprising sulfoxide functionality have a dispersity less than or equal to 1.75.

18. The carrier of claim 17 wherein the polymer is a star shaped polymer comprising the segments, a core/shell macromolecule comprising the segment or a gel comprising the segments.

19. The carrier of claim 17 further comprising cationic functionality.

20. The carrier of claim 19 wherein the segments form a hydrophilic shell around a portion including the cationic functionality.
